(19) United States
(12) Patent Application Publication
(10) Pub. No.: US 2018/0333061 A1
(43) Pub. Date: Nov. 22, 2018

(54) NEURAL MONITORING DEVICES AND METHODS

(71) Applicant: DePuy Synthes Products, Inc., Raynham, MA (US)

(72) Inventors: John Pracyk, Cumberland, RI (US); Thomas M. DiMauro, Southboro, MA (US); Michael J. O'Neil, West Barnstable, MA (US); William Horton, Duxbury, MA (US); Emir Osmanagic, Norwell, MA (US); William Kane, Newport Beach, CA (US); John Wright, Dedham, MA (US); John Canady, Iowa City, IA (US); Sean Lilienfeld, Sharon, MA (US)

(21) Appl. No.: 15/966,293

(22) Filed: Apr. 30, 2018

Related U.S. Application Data
(60) Provisional application No. 62/507,930, filed on May 18, 2017.

(51) Int. Cl. A61B 5/04; A61B 17/02; A61B 5/00

(52) U.S. Cl. CPC A61B 5/04001; A61B 17/0206; A61B 5/0086; A61B 2505/05; A61B 17/025; A61B 2017/0262; A61B 5/4041

(57) ABSTRACT

Various embodiments of a neural monitoring device and related methods are disclosed herein. An exemplary neural monitoring device can be used during various surgical procedures to assess neural activity, status, health, etc. in order to anticipate and prevent nerve damage due to neural ischemia and other neural conditions. In some embodiments, a neural monitoring device can be configured to monitor neural activity, status, health, etc. of nerves encountered during a spinal surgical procedure. Embodiments of the neural monitoring device can also be used in non-spinal surgical procedures that can risk neural damage.

### RELATED APPLICATION(S)

This application claims the benefit of U.S. Provisional Patent Application No. 62/507,930, filed on May 18, 2017, the entire contents of which are incorporated herein by reference.

### FIELD

Neural monitoring devices and related methods are disclosed herein, e.g., devices and methods for monitoring neural health using functional near infrared (fNIR) spectroscopy.

### BACKGROUND

A challenge in many types of surgery, including spinal surgeries, is avoiding injury or damage to nerves in and around a surgical site. Nerve damage can cause the patient to experience chronic pain, weakness, numbness, and/or other postsurgical neuropathy/radiculopathy. Nerve damage can be caused during surgery for a number of reasons, including neural ischemia. Neural ischemia is a condition that can cause an inadequate supply of blood flow to a nerve, resulting in a shortage of oxygen needed for neural metabolism. If a nerve is maintained in an ischemic state for too long, damage to the nerve can occur.

In some surgical procedures, neural ischemia can be caused by retracting and/or elevating a nerve or innervated tissue away from the pathway to the surgical site. When retracted, a nerve can become compressed or stretched, thereby causing a restriction in blood flow and thus oxygen to the nerve. For example, as shown in FIG. 1, during a lateral lumbar interbody fusion (LLIF) procedure, retractor blades 110 of a spinal access device 100 can be inserted into the psoas muscle 10 through an incision made in the patient's side. To form a working channel that exposes the target disc space 20, the retractor blades 110 can be operated to expand outward and retract a portion of the psoas muscle 10. However, as the psoas muscle 10 is retracted, psoas nerves 30 within the muscle, e.g., lumbar plexus nerves, can become stretched or compressed, thereby causing restriction in blood flow and thus oxygen to the nerves. If maintained in an ischemic state for too long, the psoas nerves 30 can become damaged due to a shortage of oxygen needed for neural metabolism.

To prevent nerve damage due to ischemia or other causes, it can be useful to monitor nerve status or nerve health during surgery. There is a continual need for improved neural monitoring devices and related methods.

### SUMMARY

Various embodiments of a neural monitoring device and related methods are disclosed herein. An exemplary neural monitoring device can be used during various surgical procedures to assess neural activity, status, health, etc. in order to anticipate and prevent nerve damage due to neural ischemia and other neural conditions. In some embodiments, a neural monitoring device can be configured to monitor neural activity, status, health, etc. of nerves encountered during a spinal surgical procedure. Embodiments of the neural monitoring device can also be used in non-spinal surgical procedures that can risk neural damage.

In some embodiments, a device for neural monitoring can include a processor; an access device having a proximal end and a distal end and defining a working channel into a body of a patient; and a longitudinally adjustable element having a distal end and coupled to the access device. The longitudinal adjustable element can be configured to be advanced or withdrawn with respect to the distal end of the access device. One or more optodes can be disposed on the distal end of the access device and one or more counterpart optodes can be disposed on the distal end of the longitudinally adjustable element. The one or more optodes or the one or more counterpart optodes can be configured to emit NIR light and the one or more counterpart optodes or the one or more optodes can be configured to detect back-scattered NIR light. The longitudinally adjustable element can be distally advanced or withdrawn with respect to the distal end of the access device to vary a distance between the one or more optodes and the one or more counterpart optodes and thereby change the maximum depth from which to detect the back-scattered NIR light. The processor can be coupled to the one or more optodes and the one or more counterpart optodes and configured to perform neural monitoring based on the detected back-scattered NIR light.

The one or more optodes can include one or more NIR light emitters and the one or more counterpart optodes can include one or more NIR light detectors. The one or more counterpart optodes can include one or more NIR light detectors and the one or more counterpart optodes can include one or more NIR light emitters. The one or more optodes or the one or more counterpart optodes can be configured to emit a first wavelength of near infrared (NIR) light to perform neural monitoring and to emit a second wavelength of light to perform neural therapy. The second wavelength of light can be one of a red light or NIR light selected to increase the amount of adenosine triphosphate (ATP) in a nerve. The processor can be configured to determine relative concentrations of oxygenated hemoglobin and deoxygenated hemoglobin based on the detected back-scattered NIR light and monitor neural status based on the determined concentrations.

The access device can include multiple retractor blades. The one or more optodes can be disposed on the distal end of at least one of the plurality of retractor blades. The access device can be a laterally expandable tissue retractor. The longitudinally adjustable element can be a longitudinally adjustable shim moveably coupled to at least one of the retractor blades, and the one or more counterpart optodes can be disposed on the distal end of the longitudinally adjustable shim. The shim can be a distally adjustable disc anchor.

In some embodiments, a device for neural monitoring, can include a processor; an access device having a proximal end and a distal end and defining a working channel into a body of a patient; and one or more near infrared (NIR) light emitters and one or more NIR light detectors disposed on the distal end of the access device. The one or more NIR light emitters can be configured to emit NIR light and the one or more NIR light detectors can be configured to detect back-scattered NIR light. The processor can be configured to perform neural monitoring based on the detected back-scattered NIR light reflected from the nerve. The processor can be configured to determine relative concentrations of oxygenated hemoglobin and deoxygenated hemoglobin based on the detected back-scattered NIR light and monitor neural status of the nerve based on the determined concentrations. The access device can be a substantially tubular docking port.

An NIR light emitter can be configured to emit collimated NIR light into the nerve and an NIR light detector can be housed within an open-ended tube through which to detect the collimated NIR light reflected from the nerve. The one or more NIR light detectors can include multiple NIR light detectors oriented to detect the back-scattered NIR light reflected from the nerve in different directions. The processor can be configured to selectively activate one or more of the NIR light detectors to detect back-scattered NIR light reflected from the nerve in one or more of the different directions. The one or more NIR light emitters can include multiple light emitters oriented to emit NIR light towards the nerve in different directions. The processor can be configured to selectively activate one or more of the NIR light emitters to emit the NIR light towards the nerve in one or more of the different directions. At least one of the NIR light emitters can be steerable to emit the NIR light towards a nerve and at least one of the NIR light detectors can be steerable to detect the back-scattered NIR light reflected from the nerve.

In some embodiments, a device for neural monitoring can include a processor; a flexible substrate configured for placement on tissue containing one or more nerves; a longitudinally adjustable element having a distal end and configured to be advanced or withdrawn through a working channel relative to the flexible substrate; one or more optodes disposed on a surface of the flexible substrate and one or more counterpart optodes disposed on the distal end of the longitudinally adjustable element. The one or more optodes or the one or more counterpart optodes can be configured to emit near infrared (NIR) light. The one or more counterpart optodes or the one or more optodes can be configured to output a signal indicative of the emitted NIR light reflected along a path thereto. The path of the NIR light between a respective optode disposed on the flexible substrate and a respective counterpart optode disposed on the longitudinally adjustable element can be adjusted to pass through a targeted nerve or innervated tissue by distally advancing or withdrawing the longitudinally adjustable element through the working channel relative to the flexible substrate. The processor can be configured to perform neural monitoring of the targeted nerve or innervated tissue based on the signal indicative of the NIR light reflected along the path passing through the targeted nerve or innervated tissue.

The one or more optodes can include one or more NIR light emitters and the plurality of counterpart optodes comprises one or more NIR light detectors. The one or more counterpart optodes can include one or more NIR light detectors and the one or more counterpart optodes can include one or more NIR light emitters. The processor can be configured to determine based on the signal relative concentrations of oxygenated hemoglobin and deoxygenated hemoglobin associated with the targeted nerve or innervated tissue and to monitor neural status of the nerve or innervated tissue based on the determined concentrations. The longitudinally adjustable element can include a guide wire, stylet, cannula, catheter, probe, nerve retractor, or nerve shield.

In some embodiments, a device for neural monitoring can include a processor; an access device having a proximal end and a distal end and defining a working channel into a body of a patient; and an elongated instrument having a proximal end and a distal end. The elongated instrument can be configured to be inserted through the working channel of the access device into the body of the patient. One or more optodes can be disposed on the distal end of the access device and one or more counterpart optodes can be disposed on the distal end of the elongated instrument. The one or more optodes or the one or more counterpart optodes can be configured to emit near infrared (NIR) light and the one or more counterpart optodes or the one or more optodes are configured to detect back-scattered NIR light. The elongated instrument can be distally advanced or withdrawn with respect to the distal end of the access device to vary a distance between the one or more optodes and the one or more counterpart optodes and thereby change the maximum depth from which to detect the back-scattered NIR light. The processor can be coupled to the one or more optodes and the one or more counterpart optodes and configured to perform neural monitoring based on the detected back-scattered NIR light.

The processor can be configured to determine relative concentrations of oxyhemoglobin hemoglobin and deoxyhemoglobin hemoglobin based on the detected back-scattered NIR light and monitor neural status based on the determined concentrations. The one or more optodes can include one or more NIR light emitters and the one or more counterpart optodes can include one or more NIR light detectors. The one or more counterpart optodes can include one or more NIR light emitters and the one or more optodes can include one or more NIR light detectors.

The access device can be a substantially tubular docking port. The elongated instrument can be a nerve retractor having a retractor blade at the distal end. The retractor blade can have a contact surface and a non-contact surface that opposes the contact surface of the retractor blade. The contact surface of the retractor blade can be configured to contact a nerve or innervated tissue. The retractor blade can include a translucent portion between the contact surface and the non-contact surface of the blade that is translucent to NIR light. One of the contact surface and the non-contact surface of the retractor blade can be convex and one of the contact surface and the non-contact surface of the retractor blade can be concave. The one or more counterpart optodes can be one or more NIR light emitters disposed on the non-contact surface of the retractor blade such that the NIR light is emitted from the one or more NIR light emitters through the translucent portion and the contact surface of the retractor blade. The one or more counterpart optodes can be one or more NIR light detectors disposed on the non-contact surface of the retractor blade such that the back-scattered NIR light entering through the contact surface and the translucent portion of the retractor blade can be detected by the one or more NIR light detectors. The one or more counterpart optodes can be disposed in the translucent portion of the retractor blade between the contact surface and the non-contact surface of the blade. The elongated instrument can be a guide wire, stylet, cannula, catheter, probe, or nerve shield.

In some embodiments, a method for neural monitoring can include inserting an access device at least partially in an incision or a natural orifice of a patient. The access device can define a working channel having a proximal end and a distal end. One or more optodes can be disposed on the distal end of the access device. The method can further include positioning a distal end of a longitudinally adjustable element relative to the distal end of the working channel of the access device. The longitudinally adjustable element can be coupled to the access device and one or more counterpart optodes can be disposed on the distal end of the longitudinally adjustable element. The method can further include emitting near infrared (NIR) light by at least one of the optodes or counterpart optodes; outputting a signal by at least one of the counterpart optodes or optodes that is indicative of the NIR light reflected along a path thereto; advancing or withdrawing the longitudinally adjustable element below the distal end of the working channel, such that the path of the NIR light between the at least one optode disposed on the access device and the at least one counterpart optode disposed on the longitudinally adjustable element is adjusted to pass through a targeted nerve or innervated tissue; and monitoring neural status of the targeted nerve or innervated tissue based on the signal indicative of the NIR light reflected along the path passing through the targeted nerve or innervated tissue.

The one or more optodes can include one or more NIR light emitters and the one or more counterpart optodes can include one or more NIR light detectors. The one or more counterpart optodes can include one or more NIR light detectors and the one or more counterpart optodes can include one or more NIR light emitters. Monitoring neural status of the targeted nerve or innervated tissue based on the signal can include determining based on the signal relative concentrations of oxygenated hemoglobin and deoxygenated hemoglobin associated with the targeted nerve or innervated tissue; and determining the neural status associated with the targeted nerve or innervated tissue based on the determined concentrations. The access device can include multiple retractor blades and the one or more optodes can be disposed on the distal end of at least one of the retractor blades. The longitudinally adjustable element can be a distally adjustable shim coupled at least one of the retractor blades and the one or more counterpart optodes can be disposed on the distal end of the distally adjustable shim. The shim can be a distally adjustable disc anchor and the access device can be a laterally expandable tissue retractor.

In some embodiments, a method for neural monitoring can include inserting an access device at least partially in an incision or a natural orifice of a patient. The access device can define a working channel having a proximal end and a distal end. One or more near infrared (NIR) light emitters and one or more NIR light detectors can be disposed on the distal end of the access device. The method can further include emitting near infrared (NIR) light by at least one of the NIR light emitters; outputting a signal by at least one of the NIR light detectors that is indicative of the NIR light reflected along a path thereto; and monitoring a neural status of a targeted nerve or innervated tissue based on the signal indicative of the NIR light reflected along the path passing through the target nerve or innervated tissue.

Monitoring the neural status of the targeted nerve or innervated tissue based on the signal can include determining based on the signal relative concentrations of oxygenated hemoglobin and deoxygenated hemoglobin associated with the targeted nerve or innervated tissue and to determine the neural status of the targeted nerve or innervated tissue based on the determined concentrations. The one or more NIR light detectors can include multiple NIR light detectors oriented to receive NIR light reflected along different paths. The method can further include selectively activating one or more of the NIR light detectors to receive NIR light reflected along one or more of the different paths. The one or more NIR light emitters can include multiple light emitters oriented to emit the NIR light along different paths. The method can further include selectively activating one or more of the NIR light emitters to emit NIR light along one or more of the different paths. The access device can be a substantially tubular docking port. The method can further include controlling a direction of at least one of the NIR light emitters and at least one of the NIR light detectors to adjust the path of the NIR light reflected therebetween to pass through a targeted nerve or innervated tissue.

In some embodiments, a method for neural monitoring can include inserting a flexible substrate through an incision or a natural orifice of a patient and placing the flexible substrate on tissue containing one or more nerves and positioning a distal end of a longitudinally adjustable element relative to the flexible substrate. One or more optodes can be disposed on a surface of the flexible substrate and one or more counterpart optodes can be disposed on the distal end of the longitudinally adjustable element. The method can further include emitting near infrared (NIR) light by at least one of the optodes or counterpart optodes; outputting a signal by at least one of the counterpart optodes or optodes that is indicative of the NIR light reflected along a path thereto; and advancing or withdrawing the longitudinally adjustable element relative to the flexible substrate, such that the path of the NIR light between the at least one optode disposed on the flexible substrate and the at least one counterpart optode disposed on the longitudinally adjustable element can be adjusted to pass through a targeted nerve or innervated tissue; and monitoring neural status of the targeted nerve or innervated tissue based on the signal indicative of the NIR light reflected along the path passing through the targeted nerve or innervated tissue.

The one or more optodes can include one or more NIR light emitters and the one or more counterpart optodes can include one or more NIR light detectors. The one or more optodes can include one or more NIR light detectors and the one or more counterpart optodes can include one or more NIR light emitters. Monitoring the neural status of the targeted nerve or innervated tissue based on the signal can include determining based on the signal relative concentrations of oxygenated hemoglobin and deoxygenated hemoglobin associated with the targeted nerve or innervated tissue; and determining the neural status of the targeted nerve or innervated tissue based on the determined concentrations. The longitudinally adjustable element can include a guide wire, stylet, cannula, catheter, probe, nerve retractor, or nerve shield.

In some embodiments, a method for neural monitoring can include inserting an access device at least partially in an incision or a natural orifice of a patient. The access device can define a working channel having a proximal end and a distal end with one or more optodes disposed on the distal end of the access device. The method can further include inserting a distal end of an elongated instrument through working channel of the access device and below the distal end of the access device. One or more counterpart optodes can be disposed on the distal end of the elongated instrument. The method can further include emitting near infrared (NIR) light by at least one of the optodes or counterpart optodes; outputting a signal by at least one of the counterpart optodes or optodes that is indicative of the NIR light reflected along a path thereto; advancing or withdrawing the elongated instrument below the distal end of the access device, such that the path of the NIR light between the at least one optode disposed on the access device and the at least one counterpart optode disposed on the elongated instrument is adjusted to pass through a targeted nerve or innervated tissue; and monitoring neural status of the targeted nerve or innervated tissue based on the signal indicative of the NIR light reflected along the path passing through the targeted nerve or innervated tissue.

The one or more optodes can include one or more NIR light emitters and the one or more counterpart optodes can include one or more NIR light detectors. The one or more counterpart optodes can include one or more NIR light detectors and the one or more counterpart optodes can include one or more NIR light emitters. Monitoring neural status of the targeted nerve or innervated tissue based on the signal can include determining based on the signal relative concentrations of oxygenated hemoglobin and deoxygenated hemoglobin associated with the targeted nerve or innervated tissue; and determining the neural status associated with the targeted nerve or innervated tissue based on the determined concentrations. The elongated instrument can include a guide wire, stylet, cannula, catheter, probe, nerve retractor, or nerve shield. The access device can be a substantially tubular docking port.

In any of the foregoing embodiments, the targeted nerve or innervated tissue can be located at or adjacent to one or more of the spine, leg, hip, hand, shoulder, face, neck, elbow, and foot.

In some embodiments, a device for determining a state of a peripheral nerve of a patient, can include a) a retractor including a blade having thereon: i) a first near infrared light emitter, ii) a first light detector for receiving a near-infrared signal indicative of a change in light in response to directing wavelengths of light from the first near infrared light emitter onto the patient; and b) a processor for determining, from the near-infrared signal, a relative deoxyhemoglobin concentration and oxyhemoglobin concentration, and analyzing the percentage of the deoxyhemoglobin concentration to determine a corresponding state of the nerve.

The device can include multiple infrared light emitters, and the processor can be adapted to selectively irradiate the tissue contacting the retractor blade with a fluence of between 0.1 J/cm² and 100 J/cm². The device can include multiple infrared light emitters, and the processor can be adapted to selectively irradiate the tissue contacting the retractor blade with an intensity of between 1 mW/cm² and 100 mW/cm². The device can include multiple infrared light emitters, and the processor can be adapted to selectively irradiate tissue contacting the retractor blade with an intensity and fluence of infrared light sufficient to increase adenosine triphosphate (ATP) in neighboring nerve tissue. The one or more near infrared emitters can be configured to emit a first wavelength that below 810 nm and a second wavelength above 810 nm. The processor can be further adapted to analyze the percentage of the oxyhemoglobin concentration to determine a corresponding state of the nerve.

In some embodiments, the device can further include a percutaneous passageway having a proximal end and a distal end. The retractor can pass at least partially through the passageway, and the first near infrared light emitter and the first light detector can be distal of the distal end of the passageway. The percutaneous passageway can be substantially tubular and form a concave inner surface. The retractor blade can have an outer convex surface and an opposite inner concave surface. The outer convex surface of the retractor blade can face the concave inner surface of the substantially tubular passageway.

At least a portion of the retractor blade can be translucent to near infrared light, and the emitter and detector can be placed to shine through the outer convex surface of the retractor blade. The emitter and detector can be placed upon the inner concave surface of the retractor blade and oriented to shine through the outer convex surface of the retractor. At least a portion of the retractor can be translucent to near infrared light, and the emitter and detector can be placed to shine through the translucent portion of the retractor blade. The translucent portion of the retractor blade through which the near infrared light from the emitter is shined can have a convex outer surface. The emitter and detector can be placed within the retractor blade between the inner concave surface and the outer convex surface of the retractor. The emitter and detector can be placed upon the outer convex surface of the retractor blade. The retractor blade can have bilateral edges, and the emitter and detector can be placed substantially opposingly at the bilateral edges of the retractor blade. The retractor can have a proximal end and a distal end defining a longitudinal axis and the emitter and detector can be placed substantially the same distance from the distal end of the retractor. The retractor blade can be substantially transparent.

The emitter and detector can be wirelessly coupled to the processor. The emitter and detector can be coupled to the processor by hard wire. The device can have multiple infrared light emitters, and the processor can be adapted to irradiate the outer surface of the retractor blade with a fluence of between 0.1 J/cm² and 10 J/cm². The device can have multiple infrared light emitters, and the processor can be adapted to irradiate the outer surface of the retractor blade with an intensity of between 1 mW/cm² and 100 mW/cm². The device can have multiple infrared light emitters, and the processor can be adapted to irradiate the outer surface of the retractor blade with an intensity and fluence of infrared light sufficient to increase ATP in neighboring nerve tissue that substantially contacts the outer convex surface of the blade. The one or more light emitters can be configured to emit a first wavelength below 810 nm and a second wavelength above 810 nm.

In some embodiments, the retractor can be an expandable retractor having multiple blades, and at least one blade can have the first near infrared light emitter and the first infrared light detector. The processor can be adapted to analyze pulses light signals and filter out non-pulsed signals. The at least one blade can include a distally-adjustable shim, and one of the NIR emitter and NIR detector can be located on the shim. The NIR emitter can be located on the shim. The NIR detector can be located on the shim. The emitter and detector can be axially separated by a distance of between 2 cm and 7 cm. The blade can have multiple near infrared detectors. The blade can have multiple near infrared emitters. The processor can be adapted to alternately actuate the emitters. The retractor can have a proximal end and a distal end defining a longitudinal axis, and the emitter and detector can be placed at different distances from the distal end of the retractor.

In some embodiments, a method of determining a state of a nerve can include a) creating a path to the spine, b) placing a retractor blade in the path having thereon a first near-infrared light emitter and a first light detector for receiving a near-infrared signal indicative of a change in light in response to directing wavelengths of light from the first near-infrared light emitter onto the patient, c) actuating the first near infrared light emitter to emit wavelengths of infrared light into tissue adjacent the spine wherein the irradiated tissue contains the nerve, d) determining, from the near-infrared signal, a relative deoxyhemoglobin concentration and oxyhemoglobin concentration, and e) analyzing the percentage of the deoxyhemoglobin concentration to determine a corresponding state of the nerve. The tissue can be the psoas muscle. The tissue can be a nerve root.

The method can further include selectively irradiating the nerve with an intensity and fluence of infrared light sufficient to increase ATP in the nerve. The nerve can be irradiated with a fluence of between 0.1 J/cm² and 10 J/cm². The nerve can be irradiated with an intensity of between 1 mW/cm² and 100 mW/cm². The blade can have a curved distal end, and the curved distal end of the blade can be configured to contact a dural sac. The light emitter can be configured to emit a first wavelength below 810 nm and a second wavelength above 810 nm. The method can further include analyzing the percentage of the oxyhemoglobin concentration to determine a corresponding state of the nerve.

In some embodiments, a method of determining a state of a nerve can include a) creating a lateral path to the spine to expose the psoas muscle, b) placing a device upon an outer surface of the psoas muscle. The device can include a flexible component having thereon: i) a first near infrared light emitter emitting wavelengths of infrared light, ii) a first light detector for receiving a near-infrared signal indicative of a change in light in response to directing wavelengths of light from the first near infrared light emitter onto the psoas muscle; and a processor for determining, from the near-infrared signal, a relative deoxyhemoglobin concentration and oxyhemoglobin concentration, and analyzing the percentage of the deoxyhemoglobin concentration to determine a corresponding state of a nerve within the psoas muscle. The method can further include c) actuating the first near infrared light emitter to emit infrared light into tissue adjacent the spine, where the irradiated tissue containing the nerve, d) determining, from the near-infrared signal, a relative deoxyhemoglobin concentration and oxyhemoglobin concentration, and e) analyzing the percentage of the deoxyhemoglobin concentration to determine a corresponding state of the nerve. The method can further include f) analyzing the percentage of the oxyhemoglobin concentration to determine a corresponding state of the nerve. The first near infrared light emitter can be configured to emit a first wavelength below 810 nm and a second wavelength above 810 nm.

In some embodiments, a method of determining a state of the spinal cord in a scoliosis patient, can include a) creating a path to the spine to a vertebra, b) fixing a rod to the vertebra, and c) placing a device in proximity to the spinal cord. The device can include i) a first near infrared light emitter emitting wavelengths of infrared light, ii) a first light detector for receiving a near-infrared signal indicative of a change in light in response to directing wavelengths of light from the first near infrared light emitter onto the spinal cord; and iii) a processor for determining, from the near-infrared signal, a relative deoxyhemoglobin concentration and oxyhemoglobin concentration, and analyzing the percentage of the deoxyhemoglobin concentration to determine a corresponding state of the spinal cord. The method can further include d) actuating the first near infrared light emitter to emit wavelengths of infrared light into the spinal cord, e) determining, from the near-infrared signal, a relative deoxyhemoglobin concentration and oxyhemoglobin concentration, and f) analyzing the percentage of the deoxyhemoglobin concentration to determine a corresponding state of the spinal cord. The first near infrared light emitter can be configured to emit a first wavelength below 810 nm and a second wavelength above 810 nm. The method can further include f) analyzing the percentage of the oxyhemoglobin concentration to determine a corresponding state of the nerve.

In some embodiments, a method of determining a state of a compressed nerve root in a spinal patient can include a) creating a path to the spine to a vertebra and b) placing a device in proximity to the compressed nerve root. The device can include i) a first near infrared light emitter, ii) a first light detector for receiving a near-infrared signal indicative of a change in light in response to directing wavelengths of light from the first near infrared light emitter onto the spinal cord; and iii) a processor for determining, from the near-infrared signal, a relative deoxyhemoglobin concentration and oxyhemoglobin concentration, and analyzing the percentage of the deoxyhemoglobin concentration to determine a corresponding state of the spinal cord. The method can further include c) actuating the first near infrared light emitter to emit wavelengths of infrared light into the compressed nerve root, d) determining, from the near-infrared signal, a relative deoxyhemoglobin concentration and oxyhemoglobin concentration, and analyzing the percentage of the deoxyhemoglobin concentration to determine a corresponding state of the compressed nerve root, e) decompressing the nerve root, f) actuating the first near infrared light emitter to emit infrared light into the decompressed nerve root, g) determining, from the near-infrared signal, a relative deoxyhemoglobin concentration and oxyhemoglobin concentration, and h) analyzing the percentage of the deoxyhemoglobin concentration to determine a corresponding state of the decompressed nerve root. The first near infrared light emitter can include a first wavelength below 810 nm and a second wavelength above 810 nm. The method can further include analyzing the percentage of the oxyhemoglobin concentration to determine a corresponding state of the nerve.

In some embodiments, a method of determining a state of a nerve can include a) creating a path to the spine to expose a nerve root and b) placing a device at least partially in the path. The device can include a port having a distal end portion. The distal end portion can have thereon i) a first near infrared light emitter emitting wavelengths of infrared light, ii) a first light detector for receiving a near-infrared signal indicative of a change in light in response to directing wavelengths of light from the first near infrared light emitter onto the psoas muscle; and a processor for determining, from the near-infrared signal, a relative deoxyhemoglobin concentration and oxyhemoglobin concentration, and analyzing the percentage of the deoxyhemoglobin concentration to determine a corresponding state of a nerve within the psoas muscle. The method can further include c) actuating the first near infrared light emitter to emit infrared light into tissue adjacent the spine wherein the irradiated tissue contains the nerve root, d) determining, from the near-infrared signal, a relative deoxyhemoglobin concentration and oxyhemoglobin concentration, and e) analyzing the percentage of the deoxyhemoglobin concentration to determine a corresponding state of the nerve.

The first near infrared light emitter can be a laser. The first light detector can include a tube distally extending therefrom. At least one of the emitter and detector can be pivotally-adjustable. The first near infrared light emitter can be configured to emit a first wavelength below 810 nm and a second wavelength above 810 nm. The method can further comprise f) analyzing the percentage of the oxyhemoglobin concentration to determine a corresponding state of the nerve.

### BRIEF DESCRIPTION OF THE DRAWINGS

FIG. 1 is a schematic illustration of a spinal procedure performed in the lumbar region of the spine;

FIG. 2A is a schematic illustration of an exemplary embodiment of a laterally expanding tissue retractor and a movably coupled disc anchor configured for neural monitoring;

FIG. 2B is a schematic illustration of the tissue retractor of FIG. 2A in a closed configuration;

FIG. 2C is a schematic illustration of the tissue retractor of FIG. 2A in an open configuration;

FIG. 2D is a schematic illustration of the disc anchor of FIG. 2A configured for neural monitoring;

FIG. 2E is a schematic illustration of the disc anchor of FIG. 2D movably coupled to a retractor blade in an advanced position;

FIG. 2F is a schematic illustration of the disc anchor of FIG. 2D movably coupled to a retractor blade in a withdrawn position;

FIG. 2G is a schematic illustration of an exemplary configuration of NIR light emitters and detectors disposed on the retractor blades and disc anchor of the tissue retractor of FIG. 2D for neural monitoring;

FIGS. 3A and 3B are schematic illustrations of an exemplary operation of the neural monitoring device shown in FIG. 2A having one light emitting optode disposed on a retractor blade and one light detecting optode disposed on a movably coupled disc anchor or shim;

FIG. 4A is a schematic illustration of an exemplary operation of the neural monitoring device shown in FIG. 2A having multiple light emitting optodes disposed on a retractor blade and one light detecting optode disposed on a movably coupled disc anchor or shim;

FIG. 4B is a schematic illustration of an exemplary operation of the neural monitoring device shown in FIG. 2A having multiple light emitting optodes disposed on a retractor blade and multiple light detecting optodes disposed on a movably coupled disc anchor or shim;

FIGS. 5A and 5B are schematic illustrations of exemplary embodiments of the distal end of the disc anchor or shim shown in FIG. 2D;

FIG. 6 is a schematic illustration of an exemplary embodiment of a laterally expandable tissue retractor configured to perform neural monitoring and neural therapy;

FIGS. 7A-7F are schematic illustrations of a docking port and an elongated instrument configured for neural monitoring;

FIGS. 8A-8C are schematic illustrations of a flexible patch configured for neural monitoring;

FIGS. 9A and 9B are schematic illustrations of a docking port configured for neural monitoring;

FIGS. 10A and 10B are schematic illustrations of exemplary operation of the docking port shown in FIGS. 9A and 9B configured for neural monitoring during spinal surgery;

FIG. 11 is a schematic illustration of the docking port of FIGS. 9A and 9B having multiple light emitting optodes and multiple light detecting optodes configured for neural monitoring;

FIGS. 12A and 12B are schematic illustrations of a nerve retractor or other elongated instrument configured for neural monitoring;

FIGS. 13A-13C are schematic illustrations of a Hohmann retractor configured for neural monitoring;

FIG. 14 is a schematic illustration of exemplary components of a computing device configured to perform neural monitoring using fNIR spectroscopy; and

FIG. 15 is a schematic illustration identifying other exemplary parts of the body in which the various embodiments of the neural monitoring devices can be used.

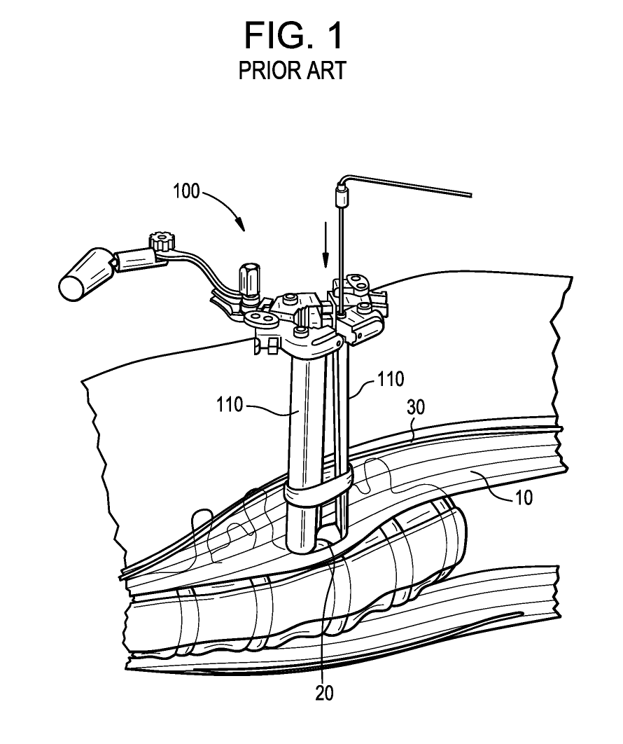

### DETAILED DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments. The features illustrated or described in connection with one exemplary embodiment may be combined with the features of other embodiments.

Functional near infrared (fNIR) spectroscopy has been used to monitor neuronal activity in the brain. FNIR spectroscopy is based on the principle of neuro-vascular coupling, sometimes referred to as the hemodynamic response or blood-oxygen-level dependent (BOLD) response. The principle of neuro-vascular coupling has been used to link neuronal activity in the brain to changes in relative concentrations of oxygenated and deoxygenated hemoglobin detected in cerebral blood flow. FNIR spectroscopy has been used to determine oxygenated and deoxygenated hemoglobin concentrations in cerebral blood flow based on absorbance measurements of near infrared (NIR) light emitted into localized regions of the brain.

FNIR spectroscopy takes advantage of an optical window of NIR light (e.g., 700-900 nm in wavelength) in which NIR light is strongly absorbed by oxygenated and deoxygenated hemoglobin in blood while more weakly absorbed by bone, skin, and other tissue. Respective concentrations of oxygenated and deoxygenated hemoglobin can also be determined based on differences in their absorption spectra. For example, oxygenated hemoglobin has greater absorption coefficients at wavelengths of NIR light above the isosbestic point of approximately 810 nm as compared to deoxygenated hemoglobin. Conversely, deoxygenated hemoglobin has greater absorption coefficients at wavelengths of NIR light below the isosbestic point as compared to oxygenated hemoglobin. Greater concentrations of oxygenated hemoglobin can be used as an indication of healthy neuronal activity, while greater concentrations of deoxygenated hemoglobin can be used as an indication of a neural ischemia. It is believed that fNIR spectroscopy can be useful in monitoring neural activity, status, health, etc. of nerves in other parts of the body.

Various embodiments of a neural monitoring device and related methods are disclosed herein for monitoring neural activity, status, health, etc. of the spinal cord, nerve roots, peripheral nerves, and/or innervated tissue (generally referred to herein as nerves and/or innervated tissue). The disclosed neural monitoring devices can be configured to monitor neural activity, status, health, etc. by using fNIR spectroscopy to monitor changes in relative concentrations of oxygenated and deoxygenated hemoglobin in localized blood flow to a nerve or innervated tissue. In some embodiments, the disclosed neural monitoring devices can be configured to adjustably target or scan specific nerves or regions of innervated tissue for neural monitoring.

An exemplary neural monitoring device can be used during various surgical procedures to assess neural activity, status, health, etc. in order to anticipate and prevent nerve damage due to neural ischemia and other neural conditions. In some embodiments, a neural monitoring device can be configured to monitor neural activity, status, health, etc. of nerves encountered during a spinal surgical procedure from a lateral approach (e.g., from a patient's side), a posterior approach (e.g., from a patient's back), or a transforaminal approach (e.g., from a patient's back outside of a facet joint). For example, such spinal procedures can include, without limitation, lumbar interbody fusions, facet injections, rhizotomy, discography, lateral and posterior laminectomy, disc clearing and cage insertion, discectomy, implant insertion, pedicle screw placement, and spinal rod placement. Embodiments of the neural monitoring device can also be used in non-spinal surgical procedures that can risk neural damage, including but not limited to thyroid surgery, hand and other extremity surgeries, trans-orifice surgery, abdominal surgery, free fibular harvesting, parotid dissection, endoscopic carpal tunnel release surgery, and revision hip surgery.

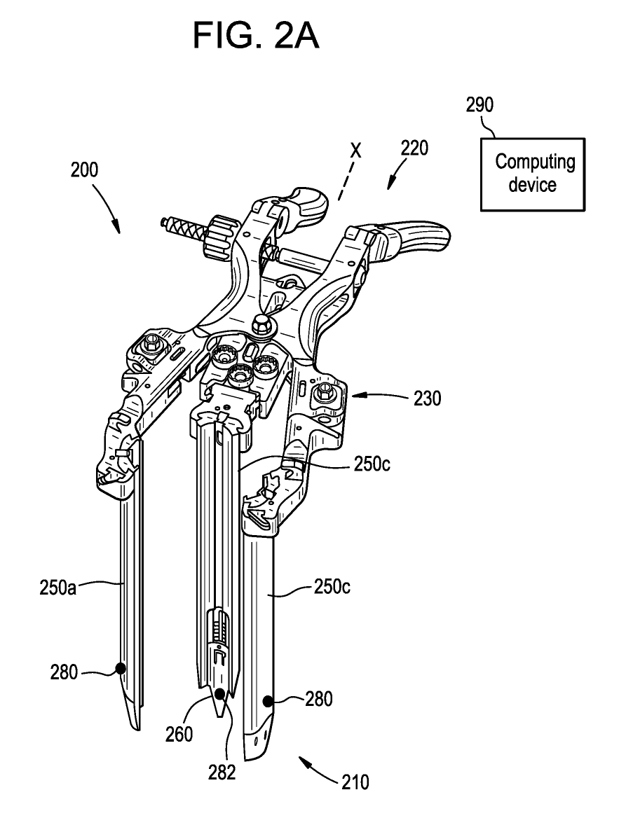

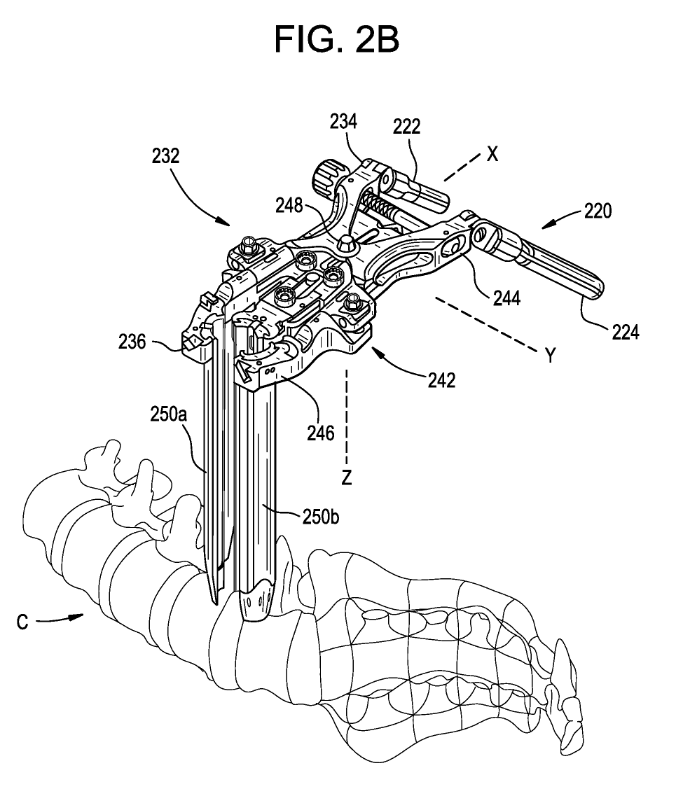

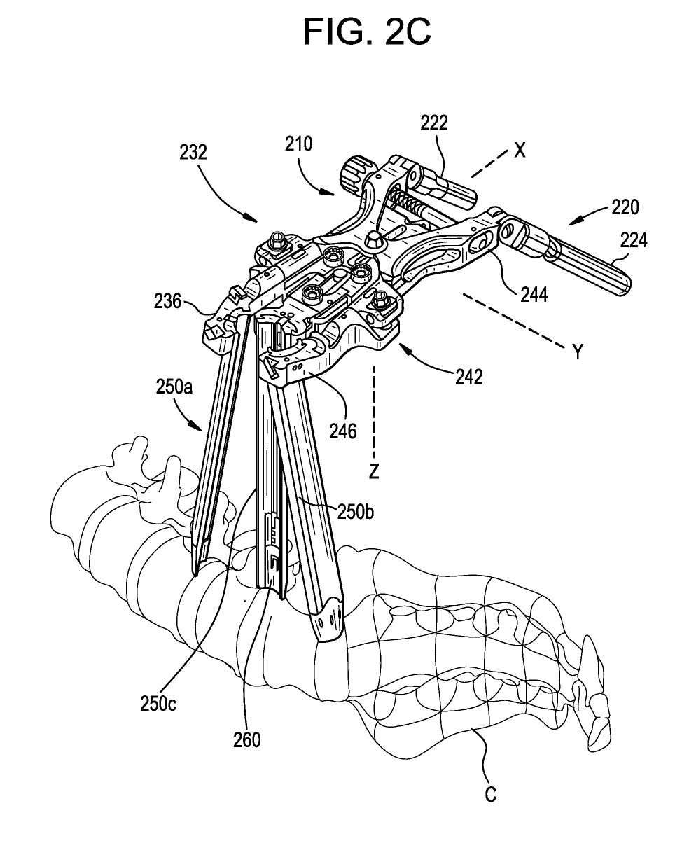

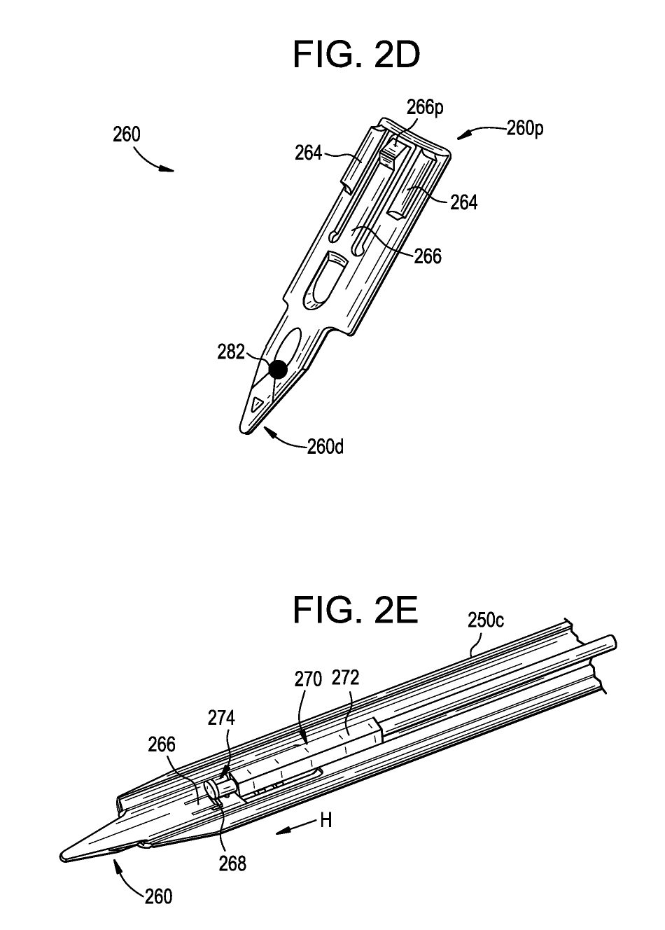

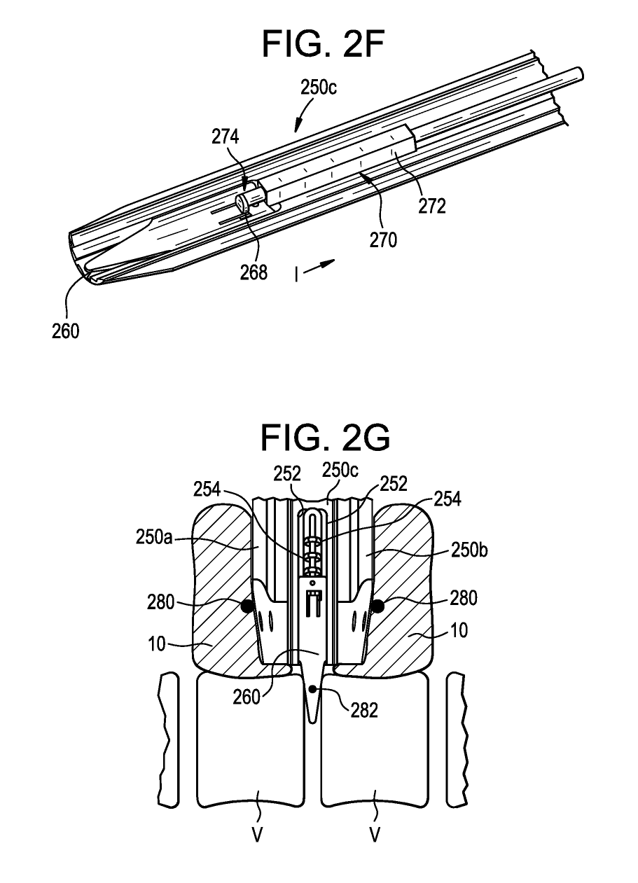

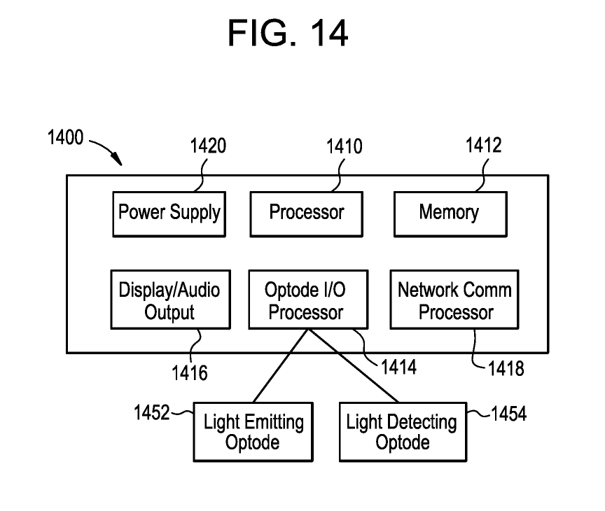

FIGS. 2A-2G are schematic illustrations of an exemplary embodiment of a neural monitoring device 200. As shown in FIG. 2A, the neural monitoring device 200 can include a laterally expanding tissue retractor 210, a disc anchor or shim 260 movably coupled to the retractor, one or more optodes 280 disposed on the retractor, one or more counterpart optodes 282 disposed on the shim, and a computing device 290 wired or wirelessly coupled to the optodes and counterpart optodes. As discussed in more detail below with respect to FIG. 14, the computing device 290 can be configured to monitor neural activity, status, health, etc. within a region of innervated tissue based on the output of a light detecting optode 280 (or 282) that is configured to detect the back-scattering of NIR light emitted into the innervated tissue region by a light emitting optode 282 (or 280).

In some embodiments, the laterally expanding tissue retractor 210 can be implemented as a spinal access retractor shown and described in U.S. patent application Ser. No. 13/237,710, filed on Dec. 20, 2011 and issued on Apr. 11, 2017, as U.S. Pat. No. 9,615,818, entitled “Spinal Access Retractor,” the entire contents of which are incorporated herein by reference. For example, as shown in FIG. 2A, the spinal access retractor 210 can include a handle assembly 220, a holder assembly 230, and three retractor blades 250a, 250b, 250c (collectively 250). However, it should be understood that the retractor 210 can have more or less than three retractor blades 250. As shown, the holder assembly 230 can be adapted to hold and support the retractor blades 250 at an orientation substantially perpendicular to a horizontal axis X. In some embodiments, the holder assembly 230 can hold the retractor blades 250 at other orientations. For example, the holder assembly 230 can hold the retractor blades 250 at an oblique angle relative to the horizontal axis X.

Irrespective of the specific retraction structure employed, one or more retractor blades 250 can move relative to the holder assembly 230 upon actuation of the handle assembly 220 or another part of the spinal access retractor 210, for retracting soft tissue (e.g., psoas muscle 10) bilaterally, unilaterally and/or angularly at or adjacent to the spinal column C. The handle assembly 220 can be operatively connected to the holder assembly 230 and include a first handle portion 222 and a second handle portion 224 each configured to the grabbed by a user.

In some embodiments, the holder assembly 230 can include first and second arms 232, 242. A first end 234 of the first arm 232 can be connected to the first handle portion 222, and a first end 244 of the second arm 242 can be connected to the second handle portion 224. A second end 236 of the first arm 232 can be configured to hold and support a retractor blade 250a, and a second end 246 of the second arm 242 can be configured to hold and support a retractor blade 250c. The first and second arms 232, 242 can be pivotally connected to each other. For example, a pivot member, such as pivot wheel or pinion 248, can pivotally couple the first arm 232 and the second arm 242.

In some embodiments, squeezing the first and second handle portions 222 and 224 together can cause the second ends 236 and 246 of the first and second arms 232, 242, respectively, to move from a first or closed position, in which the second ends are relatively close to each other, to a second or open position, in which the distal ends are spaced apart from each other. For example, as shown in FIGS. 2B and 2C, to retract tissue bilaterally, two retractor blades 250a, 250b that are oriented substantially parallel to each other can be moved simultaneously away from each other about the vertical axis Z from a first or approximated position, in which the retraction members 250a, 250b are relatively close to each other, to a second or spaced apart position, in which the retraction members are spaced apart from each other. In unilateral retraction, a first retractor blade remains stationary while a second retractor blade moves away from the first retractor blade along either the axis Y or the axis X. For angular retraction, one or more retractor blades 250 pivot relative to the rest of the spinal access retractor 210 with respect to the vertical axis Z.

A disc anchor or shim 260 can be permanently or removably coupled to any of the illustrated retractor blades 250, e.g., retractor blade 250c. As shown in the illustrated embodiment of FIG. 2D, the disc anchor 260 can have a substantially planar body that is sized and configured to slide or move along an inner surface of the retractor blade 250c. The disc anchor 260 can have a proximal end 262p and a tapered distal end 262d. The tapered distal end 262d can be adapted to penetrate an intervertebral disc. In some embodiments, the disc anchor 260 can include a retaining member 264, such as a retaining pin or deformed tab, protruding from the proximal end 262p. The retaining member 264 can be configured and sized to slide along the inner surface of the retractor blade 250c in a longitudinal groove or slot (e.g., 252 of FIG. 2G).

In some embodiments, the disc anchor 260 can include a biasing member 266 configured for engaging the indentations (e.g., 254 of FIG. 2G) defined in the inner surface of the blade 250c. For example, the biasing member 266 can be a leaf spring formed as a cutout of the disc anchor 260. However, the disc anchor 260 can include other types of biasing members. The biasing member 266 can include an engagement member 266p, such as a protrusion, that extends from a proximal end of the biasing member 266. The engagement member 266p can be sized and configured to fit within the indentations (e.g., 254) of the blade 250c to facilitate incremental distal movement of the disc anchor 260. For example, the disc anchor 260 can be configured to move along an inner surface of the retractor blade 250c between a retracted (or withdrawn) position and an extended (or advanced) position. As shown in the illustrated embodiment, the disc anchor 260 does not extend past the distal end of the retractor blade 250 in a retracted position and the disc anchor 260 can extend past the distal end of the retractor blade 250 in the extended position.

As shown in the illustrated embodiment of FIGS. 2E and 2F, the disc anchor 260 can be moved proximally and distally along the retractor blade 250c using a tool 270. For example, as shown, the tool 270 can include an elongated member 272 and a rotatable head 274. The rotatable head 274 can be configured as an eccentric wedge-shaped wheel and can fit within a slot 268 of the disc anchor 260. To move the disc anchor distally from a retracted position to an extended position, the rotatable head 274 can be inserted in slot 268 and the tool 270 can be advanced distally in the direction indicated by arrow H, as shown in FIG. 2E. The tool 270 can also be used to move the disc anchor 260 proximally in the direction indicated by arrow I from an extended position to a retracted position, as shown in FIG. 2F. For example, the rotatable head 274 can be inserted in the slot 268 and, then, the head 274 can be rotated until it lifts an engagement member 266p out of indention 254 (or any other indentation). Subsequently, the tool 270 can be advanced proximally in the direction indicated by arrow I to urge the disc anchor 260 from the extended position to the retracted position. As shown in FIG. 2G, the disc anchor 260 can be distally advanced to penetrate an intervertebral disc between two vertebrae V and thereby fix the retractor blade 250c to that intervertebral disc in order to maintain the position of the spinal access retractor 210 in relation to the patient.

As shown in FIG. 2G, the spinal access retractor 210 can be configured to facilitate neural monitoring by disposing one or more optodes 280 on at least one of the retractor blades 250 and disposing one or more counterpart optodes 282 on the longitudinally adjustable disc anchor or shim 260. In some embodiments, the optodes 280 and the counterpart optodes 282 can be NIR light emitters and NIR light detectors, respectively. In some embodiments, the optodes 280 and the counterpart optodes 282 can be NIR light detectors and NIR light emitters, respectively. For example, as shown in the illustrated embodiment, an optode 280 can be disposed on the distal end of one or more of the retractor blades 250a, 250b, and/or 250c and a counterpart optode 282 can be disposed on the distal end of the disc anchor or shim 260.

In some embodiments, multiple optodes 280 can be disposed on the distal end of one or more of the retractor blades 250a, 250b, and/or 250c and a counterpart optode 282 can be disposed on the distal end of the disc anchor or shim 260. In some embodiments, an optode 280 can be disposed on the distal end of one or more of the retractor blades 250a, 250b, and/or 250c and multiple counterpart optodes 282 can be disposed on the distal end of the disc anchor or shim 260. In some embodiments, multiple optodes 280 can be disposed on the distal end of one or more of the retractor blades 250 and multiple counterpart optode 282 can be disposed on the distal end of the disc anchor or shim 260. Alternatively or additionally, in some embodiments, one or more optodes 280 and one or more counterpart optodes 282 can be disposed on at least one of the retractor blades 250. For example, an optode 280 can be spaced apart from a counterpart optode 282 at a fixed distance along a longitudinal axis of a retractor blade 250a, 250b, and/or 250c. In some embodiments, an optode 280 can be spaced apart from a counterpart optode 282 at a fixed distance along a horizontal axis of a retractor blade 250a, 250b, and/or 250c.

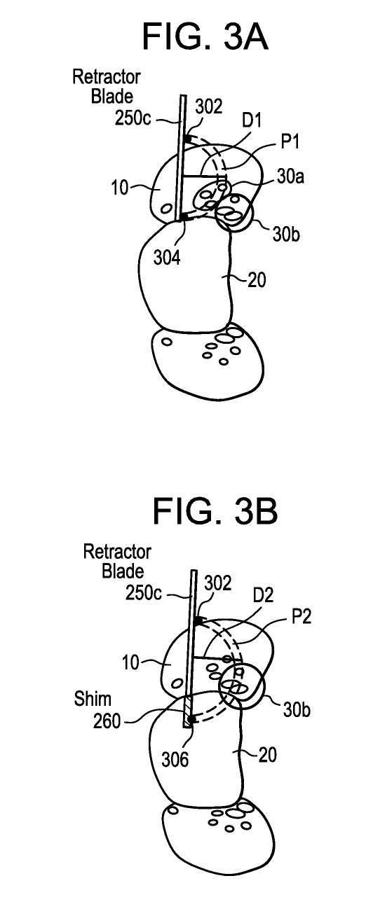

FIGS. 3A and 3B are schematic illustrations of an exemplary operation of the neural monitoring device 200 shown in FIG. 2A. As discussed in more detail with respect to FIG. 14, a computing device (e.g., 290 of FIG. 2A) can be configured to monitor neural activity, status, health, etc. within a region of innervated tissue (e.g., psoas muscle 10) based on the output of a light detecting optode. For example, as shown in FIG. 3A, a light emitting optode 302 disposed on a retractor blade (e.g., 250c) of the embodiment spinal access retractor (e.g., 200) can emit NIR light into the psoas muscle 10, while a light detecting optode 304 disposed on the blade can output a detection signal representative of back-scattered light reflected from within the psoas muscle 10.

In some embodiments, the light detecting optode 304 can detect back-scattered light that follows a generally arcuate or banana-shaped path P1 based on the spacing between the respective optodes 302, 304. For example, as shown, the light detecting optode 304 can detect back-scattered light that propagates along an arcuate path P1 having a maximum depth D1 equal to approximately one-half of the spacing between the optodes 302 and 304. Accordingly, the computing device 290 can monitor the neural activity, status, health, etc. of nerves that are located within the arcuate path P1 (e.g., psoas nerves 30a). However, when the spacing between the respective optodes 302, 304 is fixed, detection of the back-scattered light can be limited to the arcuate path P1, such that the computing device 290 can be restricted from monitoring neural activity, status, health, etc. of nerves located outside the arcuate path P1 (e.g., the psoas nerves 30b).

As discussed above, in some embodiments, a counterpart optode can be disposed on a distal end of the disc anchor or shim 260. For example, as shown in FIG. 3B, a light detecting optode 306 can be disposed on the distal end of the disc anchor 260 movably coupled to the retractor blade 250c. Thus, the distal end of the disc anchor 260 can be distally advanced or proximally withdrawn to respectively increase or decrease optode spacing, and thereby facilitate detection of back-scattered light propagating along different paths through the innervated tissue. For example, by distally advancing the distal end of the disc anchor 260 to a position below the distal end of the retractor blade 250c (e.g., into the target disc space 20), the spacing or distance between the light emitting optode 302 disposed on the blade and the light detecting optode 306 can be increased. By increasing the optode spacing, the light detecting optode 306 can detect back-scattered light that propagates along an arcuate path P2 through the psoas muscle 10 that differs from path P1. For example, the light detecting optode 306 can detect back-scattered light that following an arcuate path P2 extending to a greater maximum depth D2 and passes through psoas nerves 30b that were missed by arcuate path P1. Accordingly, the computing device 290 can selectively monitor the neural activity of nerves that are located within the arcuate paths P1 and P2 (e.g., psoas nerves 30a and 30b).

As discussed above with respect to FIG. 2G, in some embodiments, multiple optodes can be disposed on at least one of the retractor blades, multiple counterpart optodes can be disposed on a disc anchor or shim, or both multiple optodes and multiple counterpart optodes can be disposed on the retractor blades and disc anchor, respectively. In such embodiments, a computing device (e.g., 290 of FIG. 2A) can selectively activate one or more spatially different combinations of optodes and counterpart optodes to facilitate detection of NIR light back-scattered along different paths through innervated tissue (e.g. psoas muscle). Accordingly, the computing device 290 can target or scan multiple areas or regions of innervated tissue for neural monitoring.

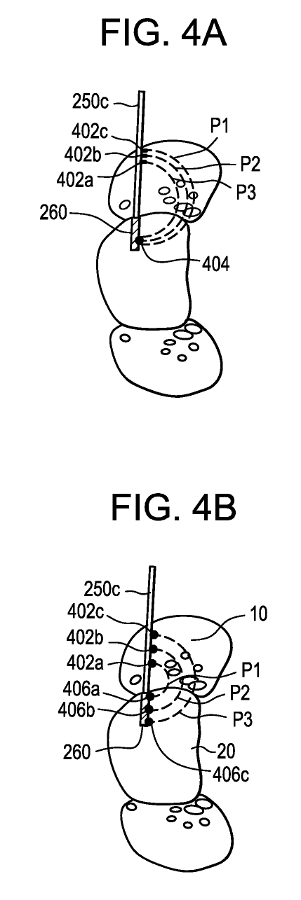

For example, FIG. 4A is schematic illustration of an operation of an exemplary embodiment of the neural monitoring device 200 shown in FIG. 2A having multiple light emitting optodes 402a, 402b, 402c (collectively 402) disposed on a retractor blade 250c and a single light detecting optode 404 disposed on a longitudinally adjustable disc anchor or shim 260. As shown, the light emitting optodes 402 can be disposed at different heights relative to the distal end of the retractor blade 250c, such that the spacing or distance differs between the light detecting optode 404 and each of the light emitting optodes 402. Although three light emitting optodes 402 are shown, it should be understood that two or more light emitting optodes 402 can be disposed on the blade.

By selectively activating one of the light emitting optodes (e.g., 402a, 402b, or 402c), the light detecting optode 404 can detect NIR light back-scattered along a respective path based on the spacing or distance between the activated light emitting optode and the light detecting optode 402. For example, by activating the light emitting optode 402a on the blade, the light detecting optode 404 disposed on the shim 260 can detect NIR light back-scattered along a first arcuate path P1 through psoas muscle. By activating light emitting optode 402b on the blade, the light detecting optode 404 can detect NIR light back-scattered along a second arcuate path P2 that is different than the first arcuate path P1. Similarly, by activating light emitting optode 402c on the blade, the light detecting optode 404 can detect NIR light back-scattered along a third arcuate path P3 that is different from the first and second arcuate paths P1 and P2. In some embodiments, there may be some overlap between the paths. In some embodiments, more than one of the light emitting optodes 402 can be selectively activated at one time.

FIG. 4B is schematic illustration of an operation of an exemplary embodiment of the neural monitoring device shown in FIG. 2A having multiple light emitting optodes 402a, 402b, 402c (collectively 402) disposed on a retractor blade 250c and multiple light detecting optodes 406a, 406b, 406c (collectively 406) disposed on a longitudinally adjustable disc anchor or shim 260. As shown in the illustrated embodiment, the light emitting optodes 402 can be disposed at different heights relative to the distal end of the retractor blade 250c, and the light detecting optodes 406 can be disposed at different heights relative to the distal end of the disc anchor 260. Accordingly, the spacing or distance between respective pairs of the light emitting optodes 402 and the light detecting optodes 406 can differ. Although three light emitting optodes 402 and three light detecting optodes 406 are shown, it should be understood that two or more light emitting optodes 402 and two or more light emitting optodes 406 can be disposed on the blade and disc anchor respectively.

By selectively activating at least one of the light emitting optodes 402 and at least one of the light detecting optodes 406, NIR light back-scattered can be detected along different paths based on the spacing or distance between the activated optodes. For example, by activating the light emitting optode 402a on the retractor blade 250c and the light detecting optode 406a on the disc anchor 260, the light detecting optode 406a can detect NIR light back-scattered along a first arcuate path P1 through psoas muscle. Similarly, by activating light emitting optode 402b on the blade and the light detecting optode 406b on the anchor, NIR light back-scattered can be detected along a second arcuate path P2 with a greater maximum depth generally concentric to the first arcuate path P1. Similarly, by activating light emitting optode 402c on the blade and the light detecting optode 406c on the anchor, NIR light back-scattered can be detected along a third arcuate path P3 with a greater maximum depth generally concentric to the first and second arcuate paths P1 and P2.

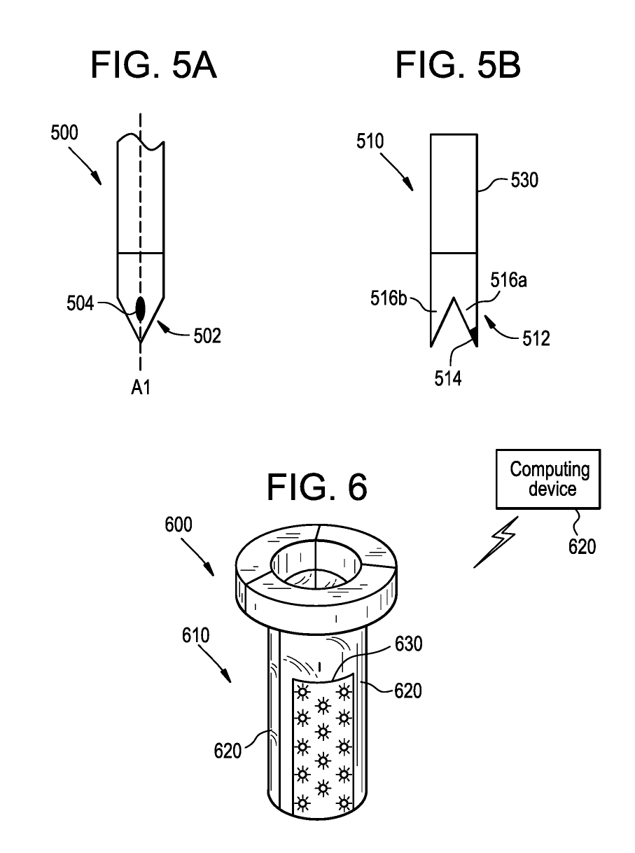

FIGS. 5A and 5B are schematic illustrations of exemplary embodiments of the distal end of a disc anchor or shim. For example, as shown in FIG. 5A, the disc anchor or shim 500 can have a V-shaped or tapered distal end 502. The tapered distal end 502 of the disc anchor 500 can facilitate insertion of the anchor into narrow spaces, such as a target disc space between adjoining vertebrae, e.g., as shown in FIG. 2G. In some embodiments, one or more counterpart optodes 504 (e.g., light emitters and/or detectors) can be disposed at or near the vertex of the tapered distal end 502 along a central axis A1 of the anchor 500. In some embodiments, the optodes 504 can be disposed on either side of the central axis A1 of the anchor.

In some embodiments, the disc anchor or shim 510 can have a bicuspid distal end 512. For example, as shown in FIG. 5B, the bicuspid distal end 512 of the anchor can have an inverted-V shape, such that at least one counterpart optode 514 (e.g., light emitter or detector) can be disposed at or near a vertex of at least one of the bicuspids 516a, 516b. By disposing an optode 514 at a bicuspid (e.g., 516a), the optodes can be positioned laterally closer to the nerves (e.g., psoas nerves 10) adjacent to at least one lateral side 530 of the anchor 510, and thereby increase the reach, or depth of analysis, within the psoas muscle or other innervated tissue region.

FIG. 6 is a schematic illustration of an exemplary embodiment of a neural monitoring device 600 configured to perform neural therapy. In some embodiments, the neural monitoring device 600 can include a spinal access retractor 610 wired or wirelessly coupled to a computing device 620 and configured to perform neural therapy. The spinal access retractor 610 can include two or more retractor blades 620 having one or more light emitters 630 disposed on at least one of the blades and configured to emit light for neural therapy. In some embodiments, the neural therapeutic light emitters 630 can be controlled by the computing device 620.

In some embodiments, the neural monitoring device 600 can have the same or similar structure and operation as the neural monitoring device 200 of FIG. 2A. In some embodiments, the light emitters 630 can be controlled to emit light for neural monitoring and neural therapy, respectively. In some embodiments, the light emitters 630 used for neural therapy can be controlled separately from the light emitters (e.g., 280, 282 of FIG. 2G) used for neural monitoring.

In some embodiments, the neural therapeutic light emitters 630 can be configured to emit a red light or a near-infrared (NIR) light suitable for increasing the amount of adenosine triphosphate (ATP) in a nerve as shown and described in U.S. patent application Ser. No. 13/784,059, filed on Mar. 13, 2013 and issued as U.S. Pat. No. 9,480,855 on Nov. 1, 2016, entitled “NIR/Red Light For Lateral Neuroprotection,” the entire contents of which are incorporated herein by reference.

For example, in some embodiments, the neural therapeutic light emitters 630 can be configured to emit a red or NIR light having a wavelength of between about 600 nm and about 1500 nm, and more preferably between about 600 nm and about 1000 nm. In some embodiments, the neural therapeutic light emitters 630 can be configured to emit a red or NIR light having a wavelength of between about 800 nm and about 900 nm, and more preferably between about 825 nm and about 835 nm. In some embodiments, the neural therapeutic light emitters 630 can be configured to emit a red or NIR light with a fluence of between about 0.1 J/cm² and about 10 J/cm². In some embodiments, the neural therapeutic light emitters 630 can be configured to emit a red or NIR light with an intensity of between about 1 mW/cm² and about 100 mW/cm².

In some embodiments, the neural therapeutic light emitters 630 can be configured to emit a red light or an NIR light upon the psoas muscle or other innervated tissue. In some embodiments, the neural therapeutic light emitters 630 can be configured to emit red light or NIR light directly upon a nerve, nerve root, or a segment of the spinal cord itself. In some embodiments, the computing device 620 can be configured to control the neural therapeutic light emitters 630 to emit light in response to determining that such nerves or innervated tissue is in an ischemic condition.

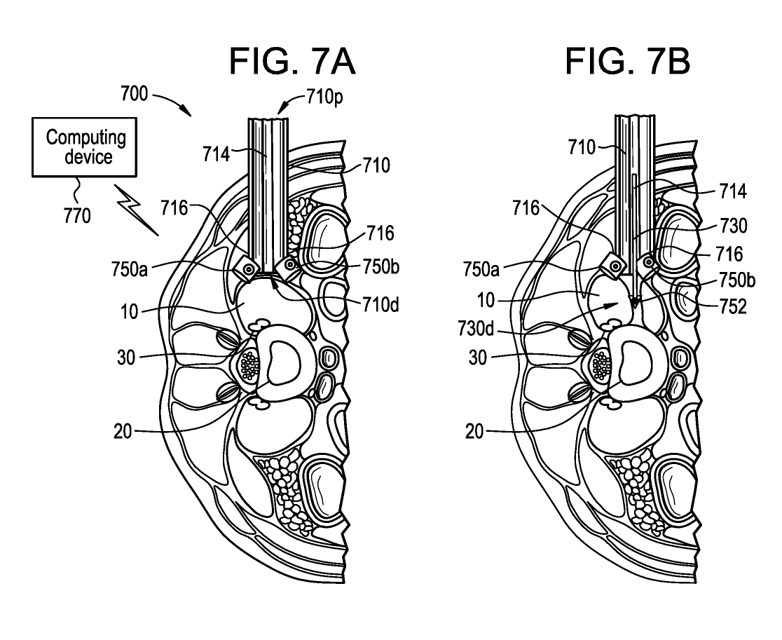

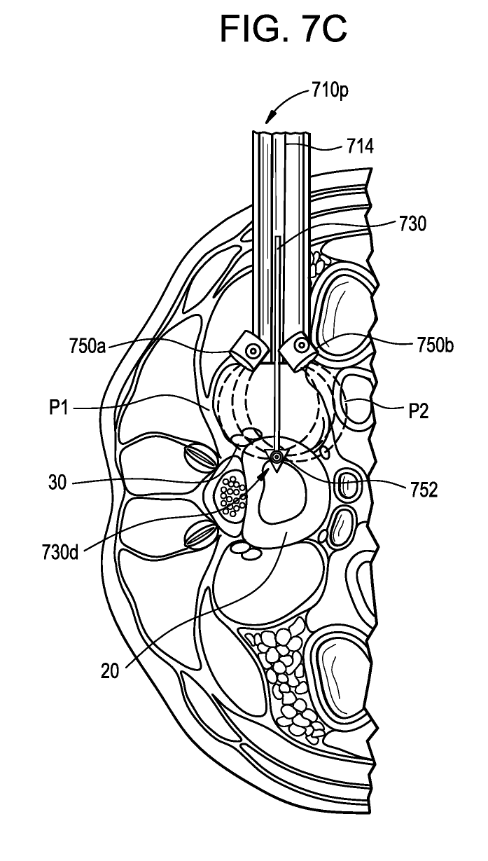

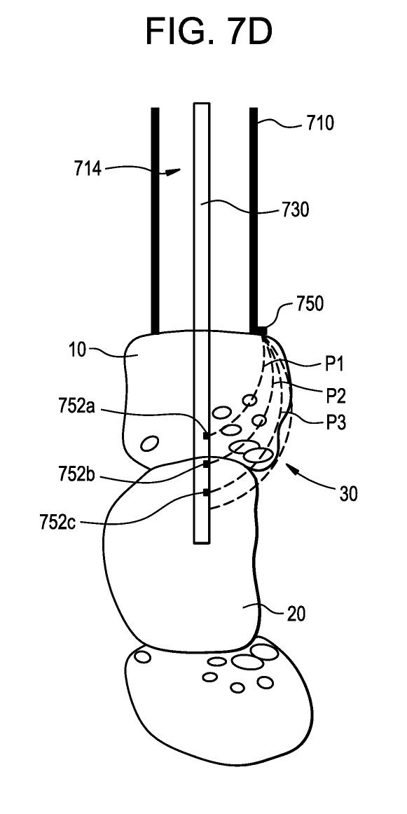

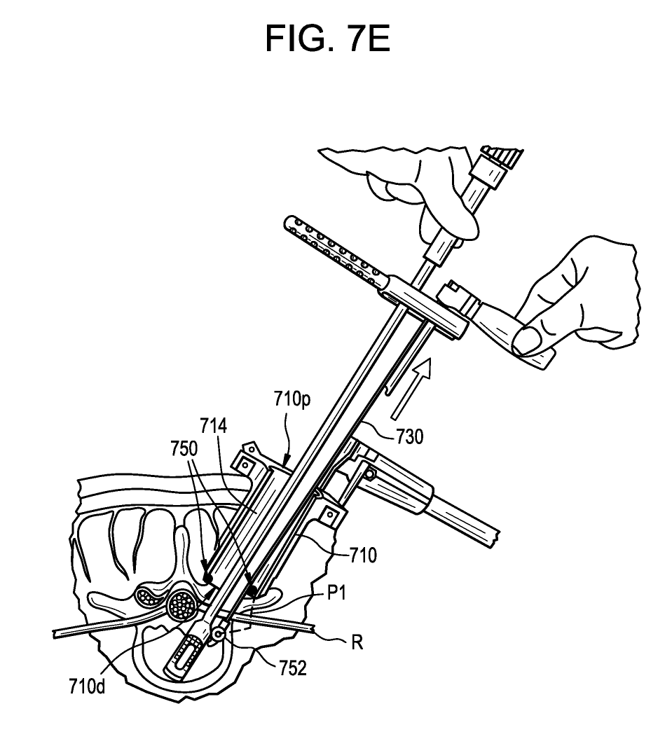

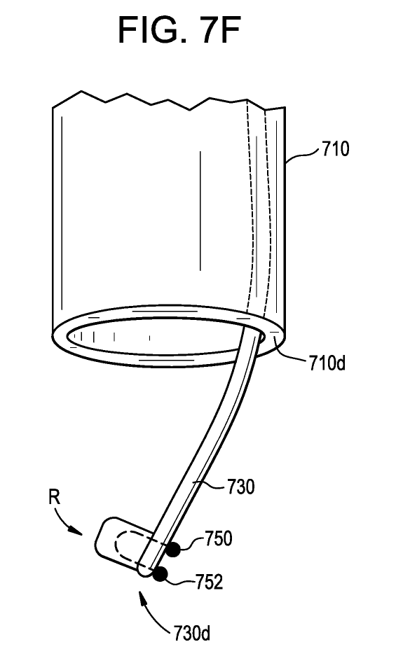

FIGS. 7A-7F are schematic illustrations of an exemplary embodiment of a neural monitoring device 700. In the illustrated embodiment, the neural monitoring device 700 can include a docking port 710, an elongated instrument 730, one or more optodes 750 disposed on the docking port 710, and one or more counterpart optodes 752 disposed on the elongated instrument 730, and a computing device 770 wired or wirelessly coupled to the optodes and counterpart optodes. As discussed in more detail below with respect to FIG. 14, the computing device 770 can be configured to determine or assess neural activity, status, health, etc. within different regions of innervated tissue based on a detection signal representative of back-scattered light output by a light detecting optode 750 (or 752) in response to emission of NIR into the innervated tissue by a light emitting optode 752 (or 750).

The docking port 710 can be used to perform surgical procedures in the spinal area including, but not limited to, lumbar interbody fusion, discectomy, implant insertion, pedicle screw placement, and spinal rod placement. However, it should be understood that the docking port 710, or various embodiments thereof, can be used in other types of surgical procedures to create an access opening or working channel that allows visibility and/or access for surgical instruments to a surgical site in the patient's body. In some embodiments, the docking port 710 can include an access device shown and described in U.S. patent application Ser. No. 15/254,877, filed on Sep. 1, 2016, entitled “Multi-Shield Spinal Access System,” and published as U.S. Publication No. 2017/0065269, the entire contents of which are incorporated herein by reference.

The docking port can be a cannula, retractor, or other access device. The docking port can have a fixed outer dimension or diameter, or can be selectively expandable and/or collapsible. As shown in the illustrated embodiments, the docking port 710 can include a body having an open proximal end 710p and an open distal end 710d that define a working channel 714 there between to facilitate percutaneous access to a surgical site. The body of the docking port 710 can have a tubular or other hollow shape to facilitate insertion of various medical instruments, including but not limited to the elongated instrument 730, into the open proximal end 710p and through the open distal end 710d. In some embodiments, the elongated instrument 730 can include a guide wire, stylet, cannula, catheter, probe, nerve retractor, nerve shield, or other elongated surgical instrument.

To facilitate neural monitoring using fNIR spectroscopy, one or more optodes 750 can be disposed at the distal end 710d of the docking port 710 and one or more counterpart optodes 752 can be disposed on the distal end 730d of the elongated instrument 730. For example, as shown in the illustrated embodiment, two optodes 750a, 750b (collectively 750) can be diametrically disposed on or adjacent to a distal-facing rim 716 of the docking port 710, and a single counterpart optode 752 can be disposed at the distal end 730d of the elongated instrument 730. It should be understood, however, that more than two optodes 750 can be disposed at the distal end 710d of the docking port 710, such that multiple optodes 750 can be spaced apart along the entire circumference of the distal-facing rim 716 of the docking port 710. In some embodiments, more than one counterpart optode 752 can be disposed at the distal end 730d of the elongated instrument 730, such that multiple counterpart optodes 752 can be spaced apart longitudinally along the distal end 730d of the elongated instrument 730.

In some embodiments, the optodes 750 disposed on the docking port 710 can be configured with a fixed orientation, such that the optodes can emit or detect NIR light in a specific direction. In some embodiments, the optodes 750 disposed on the docking port 710 can be configured to have an adjustable orientation, such that the direction in which each optode emits or detects NIR light can be changed manually or programmatically. In some embodiments, the optodes 750 disposed on the docking port 710 can be configured to scan across a range of directions while concurrently emitting or detecting NIR light.

As shown in FIGS. 7A-7C, embodiments of the neural monitoring device 700 can be used to perform neural monitoring during a spinal procedure using a lateral approach (e.g., from a patient's side). For example, referring to FIG. 7A, the docking port 710 can be inserted through an incision made in the patient's side until the distal end of the port abuts the psoas muscle 10 disposed directly lateral to a target disc space. After fixing the docking port 710 in place, the elongated instrument 730 of the neural monitoring device 700 can be inserted into the open proximal end 710p and distally advanced through the working channel 714 and out the open distal end 710d of the docking port 710. The elongated instrument 730 can be further inserted to extend into the psoas muscle 10 (as shown in FIG. 7B) or through the psoas muscle 10 into the target disc space 20 (as shown in FIG. 7C).

In some embodiments, the optodes 750 disposed on the docking port 710 and the counterpart optodes 752 disposed on the elongated instrument 730 can be NIR light emitters and NIR light detectors, respectively. The light emitting optodes 750a and 750b can be selectively activated, e.g., individually or in groups, to emit NIR light into the psoas muscle 10, while the light detecting counterpart optode 752 disposed on the elongated instrument 730 detects the back-scattered light that propagates along a respective arcuate or banana-shaped path. For example, when the light emitting optode 750a is activated to emit NIR light, the light detecting counterpart optode 752 can detect back-scattered light that propagates along a first arcuate-shaped path P1 through the innervated tissue. Similarly, when the light emitting optode 750b is activated to emit NIR light, the light detecting counterpart optode 752 can detect back-scattered light that propagates along a second or different arcuate-shaped path P2 through the innervated tissue. The light detecting optodes 752 can, in turn, output a detection signal representative of the detected back-scattered light to the computing device 770 for assessing neural activity, status, health, etc.

In some embodiments, the optodes 750 disposed on the docking port 710 and the counterpart optodes 752 disposed on the elongated instrument 730 can be NIR light detectors and NIR light emitters, respectively. For example, the light emitting counterpart optode 752 disposed on the elongated instrument 730 can be configured to emit NIR light into the psoas muscle 10, and the light detecting optodes 750a and 750b disposed on the docking port 710 can be configured to detect the back-scattered light that propagates along respective arcuate or banana-shaped paths (e.g., paths P1 and P2) from the light emitting counterpart optode 752. The light detecting optodes 750 can each output a respective signal representative of the detected back-scattered light to the computing device 770 for assessing neural activity, status, health, etc.

By distally advancing or withdrawing the elongated instrument 730, the distance separating the optodes 750 and the counterpart optodes 752 can be changed and thereby enable the light detecting optodes to detect back-scattered light propagating along different paths through the psoas muscle 10 or other innervated tissue. Accordingly, by controlling the separation between the optodes and counterpart optodes and thus the detection of back-scattered light along different paths through the innervated tissue, multiple areas or regions of innervated tissue can be targeted or scanned for neural monitoring.

As shown in FIG. 7D, in some embodiments, multiple counterpart optodes can be disposed in a spaced configuration along the elongated instrument 730. For example, as shown in the illustrated embodiment, multiple counterpart optodes 752a, 752b, 752c (collectively 752) can be disposed longitudinally spaced apart along the distal end 730d of the elongated instrument 730. When one of the optodes 750 disposed on the docking port 710 emits NIR light into the psoas muscle, each of the respective light detecting counterpart optodes 752 can detect back-scattered light propagating along a respective arcuate path (e.g., path P1, P2, or P3) through the psoas muscle. In some embodiments, the light detecting counterpart optodes 752 can be simultaneously activated to detect back-scattered light propagating along the respective arcuate paths. In some embodiments, the counterpart optodes 752 can be sequentially activated to detect back-scattered light propagating along the respective arcuate paths. Conversely, in some embodiments, the counterpart optodes 752 disposed on the elongated instrument 730 can be configured as light emitting optodes, which can be selectively activated to emit NIR light into the psoas muscle 10 or other innervated tissue and detected by one or more of the light detecting optodes 750 disposed on the docking port 710.

As shown in FIG. 7E, embodiments of the neural monitoring device 700 can also be used to perform neural monitoring during a spinal procedure using a transforaminal approach. For example, referring to FIG. 7E, the docking port 710 can be inserted through an incision made in the patient's back outside a facet joint. After fixing the docking port 710 in place, the elongated instrument 730 can be inserted into the open proximal end 710p and distally advanced through the working channel 714 and out the open distal end 710d of the docking port 710. The elongated instrument 730 can be distally advanced through a space between the facet joint and into the target disc space 20 to facilitate neural monitoring of a nerve (e.g., nerve root R) using the optodes 750 and counterpart optodes 752 in the manner disclosed above with respect to FIGS. 7A-7D. In some embodiments, the distal end 730d of the elongated instrument 730 can be configured with a blade or shield to respectively retract or shield the exiting nerve root R, e.g., during a TLIF procedure through Kambin's triangle or other surgical procedure using transforaminal approach, while concurrently monitoring the neural activity, status, health, etc. of the nerve root.

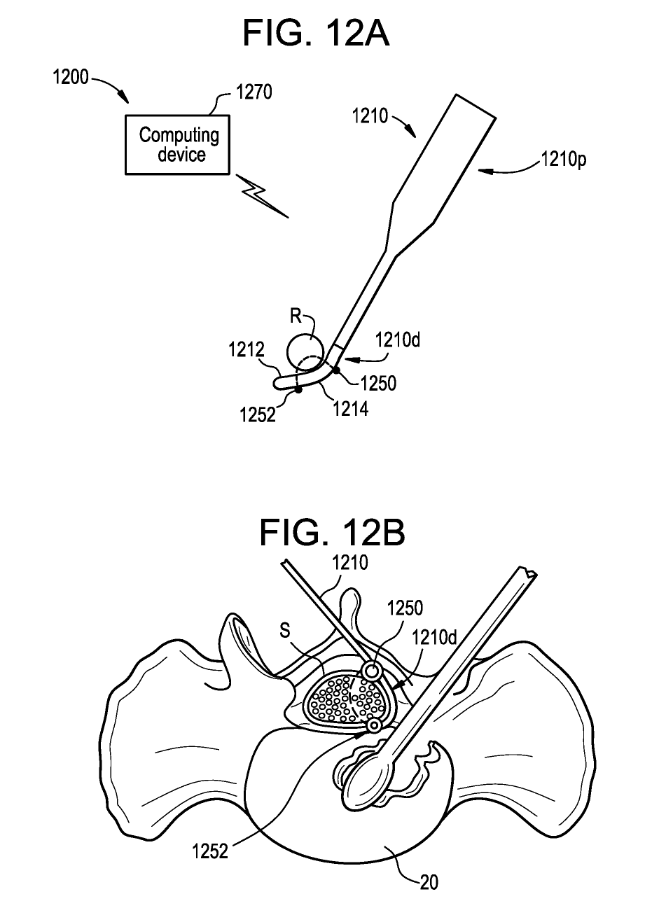

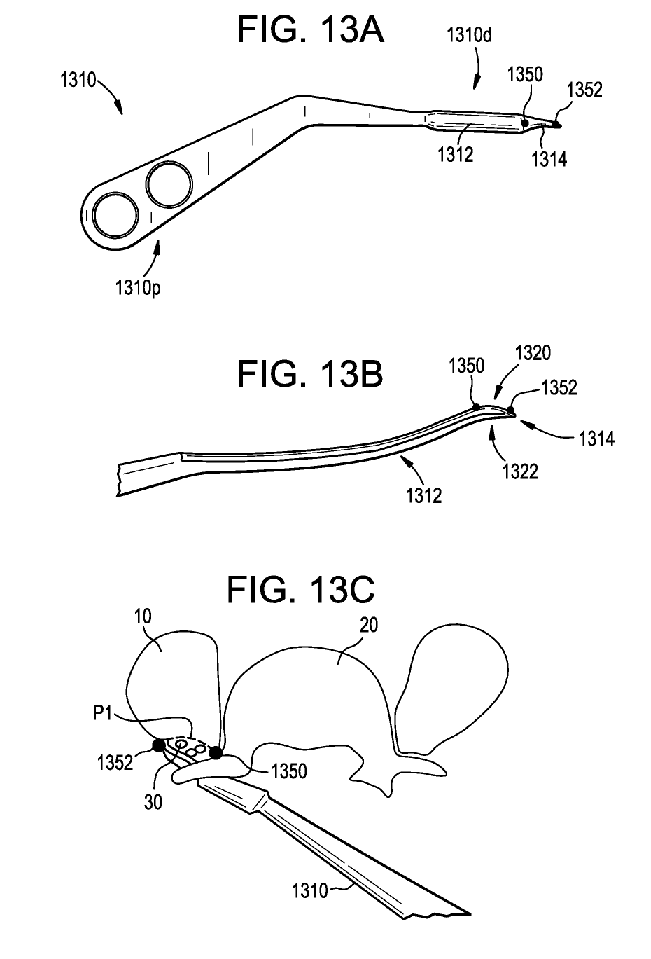

Although FIGS. 7A-7E illustrate the optodes 750 and the counterpart optodes 752 being disposed on the docking port 710 and the elongated instrument 730, other arrangements of the optodes 750, 752 are contemplated. For example, as shown in FIG. 7F, the one or more optodes 750 and the one or more counterpart optodes 752 can be disposed on the distal end 730d of the elongated instrument 730. In such embodiments, a nerve, such as an exiting nerve root R, adjacent to or in contact with the blade or shield can monitored. In some embodiments, the body of the elongated instrument 730 or a portion of the blade or shield at its distal end 730d can be translucent, such that the optodes 750, 752 can be embedded within or disposed on an opposing side of the blade or shield that avoids direct contact with the nerve, and thereby reduces nerve irritation. In some embodiments, the optodes 750 and counterpart optodes 752 can be disposed on the distal end 710d of the docking port 710. In such embodiments, a nerve encountered in the path of the NIR light between an optode 750 and counterpart optode 752 can be monitored. In some embodiments, the elongated instrument 730 can be configured to have a structure the same as or similar to the structure of the nerve retractor 1210 and/or 1310 shown and described in connection with FIGS. 12A, 12B, and 13A-13C and having at least one optode, at least one counterpart optode, or at least one optode and counterpart optode.

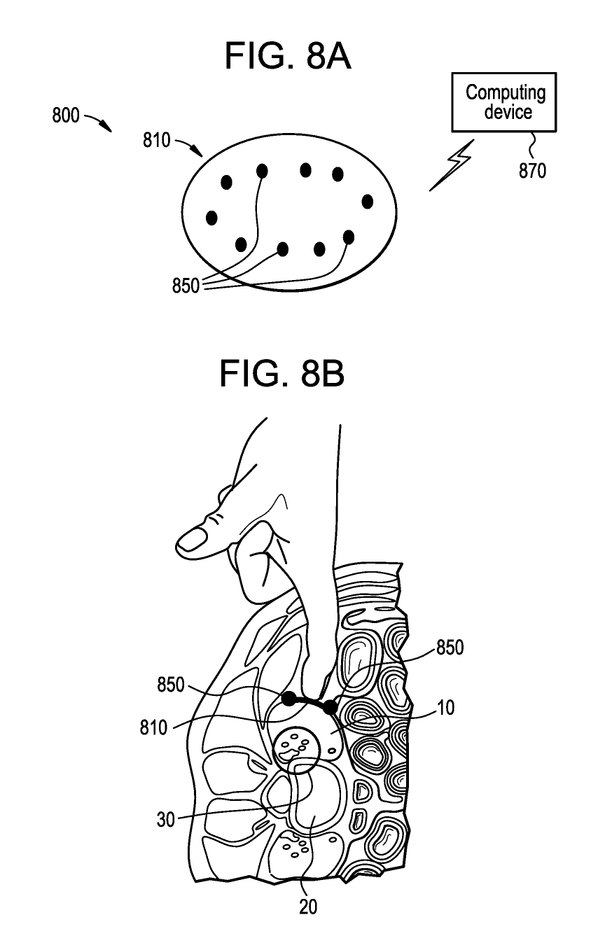

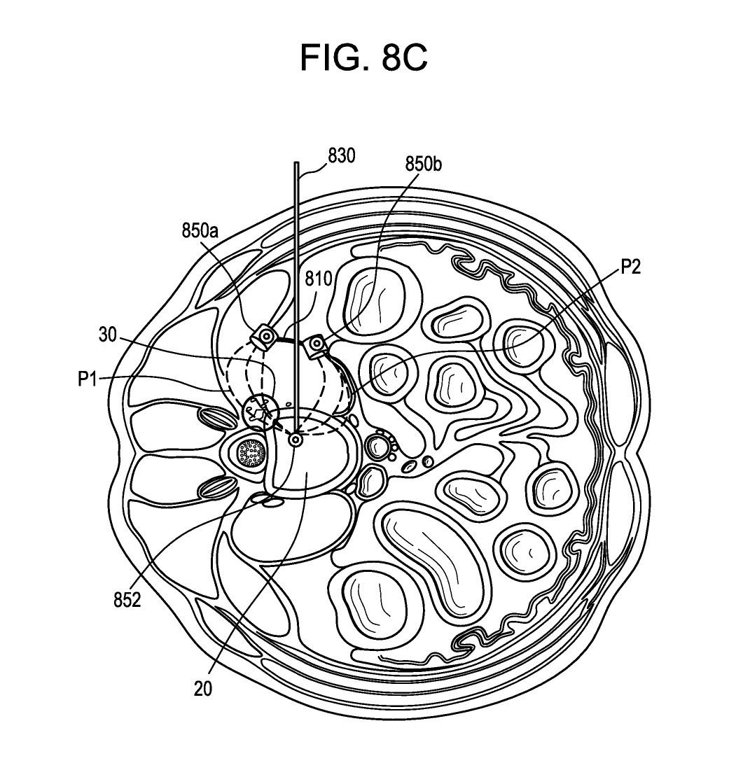

FIG. 8A-8C are schematic illustrations of an exemplary embodiment of a neural monitoring device 800. As shown in the illustrated embodiment, the neural monitoring device 800 can include a flexible substrate or patch 810, an elongated instrument 830, one or more optodes 850 disposed on the patch, one or more counterpart optodes 852 disposed on the elongated instrument, and a computing device 870 wired or wirelessly coupled to the optodes and counterpart optodes. As discussed in more detail below with respect to FIG. 14, the computing device 870 can be configured to determine or assess neural activity, status, health, etc. within different regions of innervated tissue based on a detection signal representative of back-scattered light output by a light detecting optode 850 (or 852) in response to emission of NIR into the innervated tissue by a light emitting optode 852 (or 850).

As discussed below, in some embodiments, the optodes 850 and counterpart optodes 852 can be disposed on the same patch 810 to monitor neural activity, status, health, etc. of nerves. In such embodiments, use of the elongated instrument 830 having one or more counterpart optodes disposed thereon can be optional. In some embodiments, an onboard battery or other power supply mechanism can be disposed on or embedded within the patch 810 to supply power to the optodes 850 and/or 852 disposed on the patch. In some embodiments, the optodes 850 and/or 852 disposed on the patch 810 can be coupled to an external power supply and/or the computing device 870 or other controller by a tether or cable.

In some embodiments, the neural monitoring patch 810 can be a flexible biocompatible polymer or other material sized and shaped to facilitate percutaneous insertion and deployment within a patient. For example, as shown in the illustrated embodiment, the neural monitoring patch 810 can be formed in an elliptical shape suitable for wrapping about a portion of the psoas muscle 10. In some embodiments, the neural monitoring patch 810 can be formed in other geometrical shapes suitable for application to a mass of innervated tissue. In some embodiments, the elongated instrument 830 can be a guide wire, stylet, cannula, catheter, probe, nerve retractor, nerve shield or other elongated surgical instrument.

To facilitate neural monitoring using fNIR spectroscopy, one or more optodes 850 can be disposed on or embedded in the neural monitoring patch 810 and one or more counterpart optodes 852 can be disposed at the distal end 830d of the elongated instrument 830. For example, as shown in FIG. 8A, multiple optodes 850 can be disposed in a ring-shaped or other geometrical pattern on the patch 810 and a single counterpart optode 852 can be disposed at the distal end 830d of the elongated instrument 830. In some embodiments, the optodes 850 and the counterpart optodes 852 can be NIR light emitters and NIR light detectors, respectively. Conversely, in some embodiments, the optodes 850 and the counterpart optodes 852 can be NIR light detectors and NIR light emitters, respectively.

In some embodiments, the optodes 850 disposed on the patch 810 can be configured with a fixed orientation, such that the optodes can emit or detect NIR light in a specific direction. In some embodiments, the optodes 850 disposed on the patch 810 can be configured to have an adjustable orientation, such that the direction in which each optode emits or detect NIR light can be changed manually or programmatically. In some embodiments, the optodes 850 disposed on the patch 810 can be configured to scan across a range of directions while concurrently emitting or detecting NIR light.

In some embodiments, the neural monitoring device 800 can be used to perform neural monitoring during surgical procedures in the spinal area including, but not limited to, lumbar interbody fusion, discectomy, implant insertion, pedicle screw placement, and spinal rod placement. For example, as shown in FIGS. 8A-8C, embodiments of the neural monitoring device 800 can be used to perform neural monitoring during a spinal procedure using a lateral approach (e.g., from a patient's side). However, it should be understood that the neural monitoring device 800, or various embodiments thereof, can be used in other types of surgical procedures to monitor the neural activity, status, health, etc. within innervated tissue at or about a surgical site in the patient's body.

An exemplary operation of the neural monitoring device 800 is shown in FIGS. 8B and 8C. As shown in FIG. 8B, the neural monitoring patch 810 can be inserted through an incision made in the patient's side and wrapped about a portion of the psoas muscle 10. In some embodiments, blunt dissection can be performed using the surgeon's finger to insert and deploy the neural monitoring patch 810 onto the psoas muscle 10 directly lateral to a target disc space 20. After deploying the patch, the elongated instrument 830 can be percutaneously inserted through the incision such that the distal end 830d of the instrument is distally advanced into the psoas muscle 10 below the patch 810. For example, as shown in FIG. 8C, to facilitate neural monitoring of the psoas nerves 30 adjacent to the target disc space 20, the elongated instrument 830 can be inserted such that its distal end 730d extends through the psoas muscle 10 into the target disc space 20.

In some embodiments, the optodes 850 disposed on the patch 810 and the counterpart optode 852 disposed on the elongated instrument 830 can be NIR light emitters and NIR light detectors, respectively. The light emitting optodes 850 can be selectively activated, e.g., individually or in groups, to emit NIR light into the psoas muscle 10, while the light detecting counterpart optode 852 disposed on the elongated instrument 830 detects the back-scattered light that propagates along a respective arcuate or banana-shaped path. For example, when the light emitting optode 850a is activated to emit NIR light, the light detecting counterpart optode 852 can detect back-scattered light that propagates along a first arcuate-shaped path P1 through the innervated tissue. Similarly, when the light emitting optode 850b is activated to emit NIR light, the light detecting counterpart optode 852 can detect back-scattered light that propagates along a second or different arcuate-shaped path P2 through the innervated tissue. The light detecting optodes 852 can, in turn, output a detection signal representative of the detected back-scattered light to the computing device 870 for assessing neural activity, status, health, etc.

In some embodiments, the optodes 850 disposed on the patch 810 and the counterpart optode 852 disposed on the elongated instrument 830 can be NIR light detectors and NIR light emitters, respectively. For example, the light emitting counterpart optode 852 disposed on the elongated instrument 830 can be configured to emit NIR light into the psoas muscle 10, and the light detecting optodes 850a and 850b disposed on the patch 810 can be configured to detect the back-scattered light that propagates along respective arcuate or banana-shaped paths (e.g., paths P1 and P2) from the light emitting counterpart optode 852. The light detecting optodes 850 can each output a respective signal representative of the detected back-scattered light to the computing device 870 for assessing neural activity, status, health, etc.

By distally advancing or withdrawing the elongated instrument 830, the distance separating the optodes 850 and the counterpart optodes 852 can be changed and thereby enable the light detecting optodes to detect back-scattered light propagating along different paths through the psoas muscle 10 or other innervated tissue. Accordingly, by controlling the separation between the optodes and counterpart optodes and thus the detection of back-scattered light along different paths through the innervated tissue, multiple areas or regions of innervated tissue can be targeted or scanned for neural monitoring.

In some embodiments, the flexible substrate or patch 810 can include one or more optodes 850 and one or more counterpart optodes 852 disposed on the same surface of the patch. In such embodiments, the patch 810 can be attached to an outer surface of a patient's skin (e.g., foot or other lower extremity) in order to monitor neural activity, status, health, etc. of nerves by emitting NIR light through the light emitting optodes 850 and the computer device 870 analyzing the back-scattered light detected by the light emitting optodes 852 through the skin (e.g., foot or other lower extremity). Such embodiments can be useful to facilitate ongoing or continuous neural monitoring in diabetic patients to preempt complications associated with the disease. For example, diabetic patients can get foot problems, such as ulcers, due to loss of sensation from diabetic neuropathy. In some embodiments, embodiments of the neural monitoring patch 810 can be used externally on the patient's feet to monitor oxygen levels indicative of nerve impairment in the lower extremities, and thereby allow the clinician to recommend corrective therapies to prevent the onset of ulcers or other foot problems.

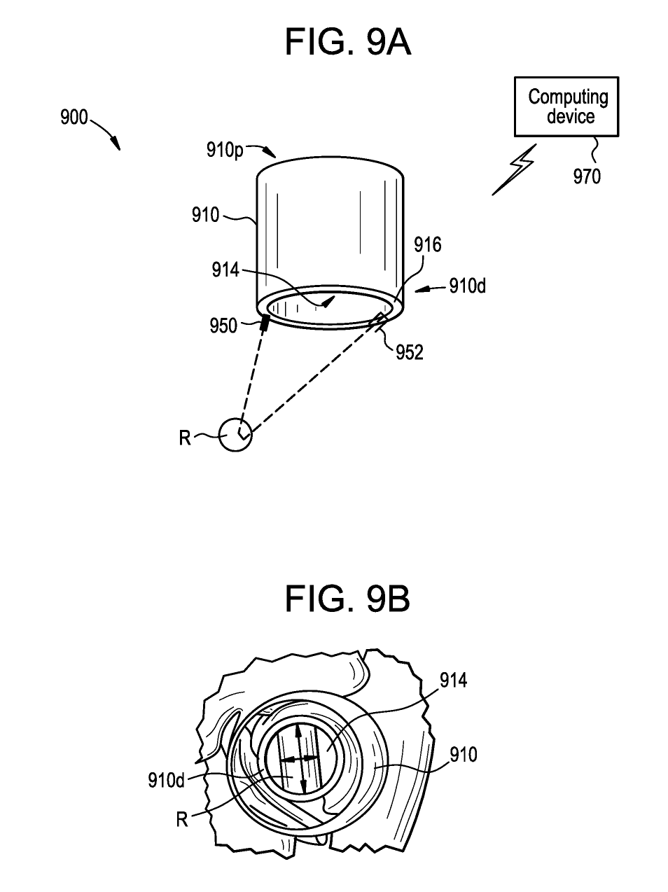

FIGS. 9A and 9B are schematic illustrations of an exemplary embodiment of a neural monitoring device 900. In the illustrated embodiment, the neural monitoring device 900 can include a docking port 910 having one or more steerable light emitting optodes 950 and one or more steerable light detecting optodes 952 disposed on a distal end 910d of the docking port 910, and a computing device 970 wired or wirelessly coupled to the optodes and counterpart optodes. As discussed in more detail below with respect to FIG. 14, the computing device 970 can be configured to determine or assess neural activity, status, health, etc. of a targeted nerve R or innervated tissue based on a detection signal representative of back-scattered light output by the light detecting optode 952 in response to the light emitting optode 950 emitting NIR light onto the targeted nerve or innervated tissue.

In some embodiments, the docking port 910 can include an access device shown and described in U.S. patent application Ser. No. 15/254,877, filed on Sep. 1, 2016, entitled “Multi-Shield Spinal Access System,” published as U.S. Publication No. 2017/0065269, the entire contents of which are incorporated herein by reference. For example, as shown in the illustrated embodiment, the docking port 910 can include a body having an open proximal end 910p and an open distal end 910d that define a working channel 914 there between to facilitate percutaneous access to a surgical site. The body of the docking port 910 can have a tubular or other hollow shape to facilitate insertion of various medical instruments into the open proximal end 910p and through the open distal end 910d.

To facilitate neural monitoring using fNIR spectroscopy, one or more steerable light emitting optodes 950 and one or more steerable light detecting optodes 952 can be disposed at the distal end 910d of the docking port 910. For example, as shown in the illustrated embodiment, a light emitting optode 950 and a light detecting optode 952 can be diametrically disposed on or adjacent to a distal-facing rim 916 of the docking port 910. It should be understood, however, that more than one light emitting optode 950 and more than one light detecting optode 952 can be disposed on the docking port 910, such that multiple light emitting and light detecting optodes can be spaced apart entirely or partially along the circumference of the distal end of the docking port. In some embodiments, the light emitting optodes 950 can be collimated light emitters configured to emit a beam of light with minimal dispersion. The light detecting optodes 952 can be housed within an open-ended tube through which to detect the collimated light. For example, such tubular light detectors can be configured to restrict detection of light to a specific direction.

In some embodiments, the light emitting optodes 950 disposed on the docking port 910 can be configured to have a steerable or adjustable orientation, such that the direction in which each optode emits NIR light can be changed manually or programmatically. In some embodiments, the light detecting optodes 952 disposed on the docking port 910 can be configured to have a steerable or adjustable orientation, such that the direction in which each optode detect NIR light can be changed manually or programmatically. For example, as shown in FIGS. 9A and 9B, in some embodiments, a light emitting optode 950 and a light detecting optode 952 can be oriented such that the optode 950 emits NIR light in a direction that targets a specific nerve 90 and the optode 952 detects back-scattered light from a direction corresponding to the targeted nerve 90. In some embodiments, a surgeon or other operator can use the computing device 970 to remotely control the orientations of the NIR light emitters and detectors. In other arrangements, the optodes 950 and/or the optodes 952 can be fixed or non-steerable. In steerable embodiments, the device 900 can include motors, linear actuators, mechanical linkages, or other structures for adjusting a position and/or orientation of the optodes 950, 952.

In some embodiments, the light emitting optode 950 can be configured to emit a visual light to serve as a visual aid when adjusting the directional orientation of the optode 950. For example, in some embodiments, the light emitting optode 950 can emit a red light capable of being seen through the working channel 914 or via a remote display as a red spot. When targeting a specific nerve 90, the surgeon or other operator can adjust the directional orientation of the light emitting optode 950 until the red spot is positioned onto the targeted nerve. In some embodiments, a separate visual light emitter co-aligned with the light emitting optode 950 can be used to provide a visual aid for adjusting the orientation of the optode.

In some embodiments, the neural monitoring device 900 can be used to perform neural monitoring during spinal surgery including, but not limited to, lumbar interbody fusion, discectomy, implant insertion, pedicle screw placement, and spinal rod placement. However, it should be understood that the neural monitoring device 900, or various embodiments thereof, can be used in other types of surgical procedures to create an access opening or working channel that allows visibility and/or access for surgical instruments to a surgical site in the patient's body.

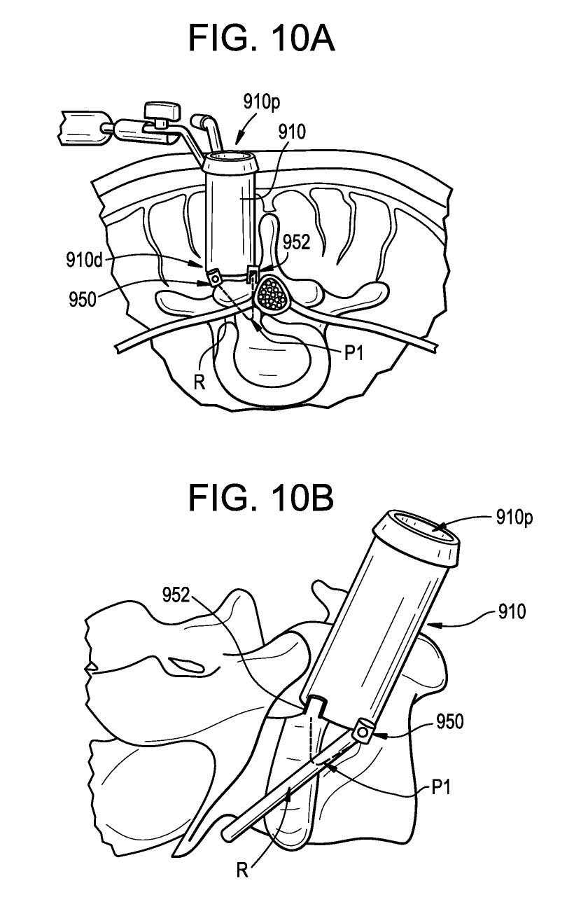

As shown in FIGS. 10A and 10B, embodiments of the neural monitoring device 900 can be used to perform neural monitoring during a spinal procedure using a transforaminal approach (e.g., from a patient's side outside a facet joint). For example, as shown in the illustrated embodiments, the docking port 910 can be inserted through an incision made in the patient's back until the distal end of the port abuts an opening, or foramen, at the side of the spine where a nerve root R exits. After fixing the docking port 910 in place, the light emitting optode 950 and the light detecting optode 952 can be remotely controlled or steered, such that the light emitting optode 950 can emit NIR light onto the target nerve root R and the light detecting optode 952 can detect NIR light back-scattered from the nerve root along a path P1. As shown in FIGS. 10A and 10B, the steerability of the optodes 950 and 952 allows specific nerves to be targeted for neural monitoring regardless of the angular orientation of the docking port 910.

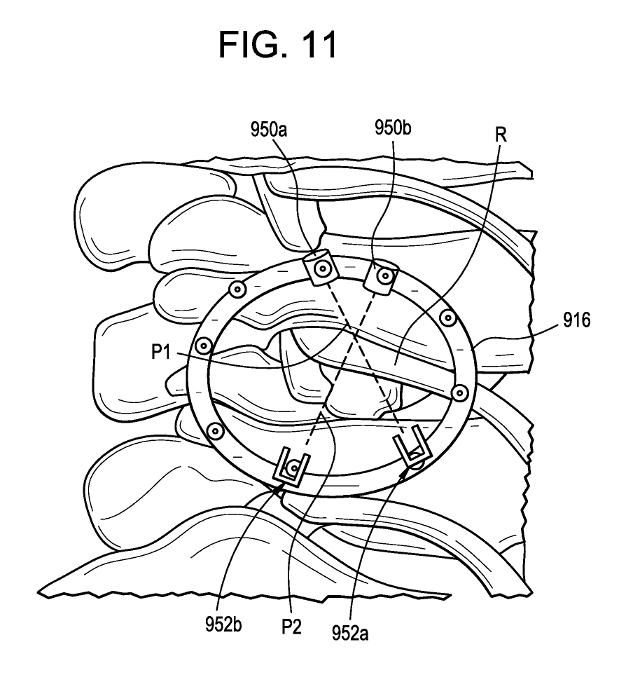

As shown in FIG. 11, multiple light emitting optodes 950 and multiple light detecting optodes 952 can be disposed along the circumference or perimeter of the distal-facing rim 916 of the docking port 910. In some embodiments, the light emitting optodes 950 and light detecting optodes 952 can be activated simultaneously to target a specific nerve or nerve root R for neural monitoring. In some embodiments, one or more pairs of the light emitting optodes 950 and light emitting optodes 950 can be sequentially or selectively activated to perform neural monitoring along different paths or directions. For example, a light emitting optode 950a can be paired with a diametrically disposed light detecting optode 952a to perform neural monitoring along a path P1, and a light emitting optode 950b can be paired with a diametrically disposed light detecting optode 952b to perform neural monitoring along a different path P2.

FIGS. 12A and 12B are schematic illustrations of exemplary embodiments of a neural monitoring device 1200. As shown, the neural monitoring device 1200 can include an elongated instrument 1210 and a computing device 1270 wired or wirelessly coupled to a light emitting optode and a light detecting optode disposed on a distal tip of the instrument. In some embodiments, the elongated instrument can include a guide wire, stylet, cannula, catheter, probe, nerve retractor, or a nerve shield. For example, in the illustrated embodiments, the elongated instrument 1210 can be a hand-held nerve retractor having a proximal handle portion 1210p and a distal retractor blade 1210d, at least one NIR light emitting optode 1250 and at least one NIR light detecting optode 1252 disposed on the retractor blade. As discussed in more detail below with respect to FIG. 14, the computing device 1270 can be configured to determine or assess neural activity, status, health, etc. of a nerve or innervated tissue based on a detection signal representative of back-scattered light output by the light detecting optode 1252 in response to the light emitting optode 1250 emitting NIR light onto the nerve or innervated tissue.

In some embodiments, the distal retractor blade 1210d, or a portion thereof, can be translucent to NIR light, such that the optodes 1250 and 1252 can be disposed on the blade in a manner that avoids direct contact with a nerve or innervated tissue during retraction. The translucent retractor blade 1210d can be configured to allow NIR light to pass through the blade from the light emitting optode 1250 onto the retracted nerve R. Similarly, the translucent retractor blade 1210d can allow back-scattered NIR light to reflect through the blade from the retracted nerve R to the light detecting optode 1252. An advantage of disposing the optodes 1250 and 1252 away from the contacting surface 1214 of the blade 1210d can include preventing nerve irritation.

For example, as shown in the illustrated embodiment of FIG. 12A, when a first surface 1212 of the translucent blade (e.g., an inner concave surface) is configured as a contact surface for retracting a nerve R, the light emitting optode 1250 and the light detecting optode 1252 can be disposed on a second surface 1214 of the blade (e.g., an outer convex surface) that opposes the first surface. In some embodiments, when the second surface 1214 of the translucent blade (e.g., the outer convex surface) is configured as the contact surface for retracting the nerve R, the light emitting optode 1250 and the light detecting optode 1252 can be disposed on the first surface 1212 of the blade (e.g., an inner concave surface) opposite the second surface 1214. In some embodiments, the light emitting and light detecting optodes 1250, 1252 can be disposed or embedded between the first and second surfaces 1212, 1214, such that both optodes avoid direct contact with a retracted nerve or innervated tissue.

Embodiments of the neural monitoring device 1200 can be used during various surgical procedures to assess neural activity, status, health, etc. in order to anticipate and prevent nerve damage due to neural ischemia and other neural conditions. For example, in the illustrated embodiment of FIG. 12B, the neural monitoring device 1200 can be used to perform neural monitoring on a portion of the spinal cord S during a surgical procedure using a posterior approach, such as a posterior lumbar interbody fusion (PLIF) procedure. As shown, the hand-held nerve retractor 1210 can be inserted through an incision made in the patient's back and distally advanced through a space created by removal of lamina adjacent to the spinal cord S. The retractor 120 can be manipulated to retract the spinal cord S to one side, or to shield the spinal cord S without retraction, thereby enabling the surgeon to clean the disc space 20 of disc material and insert bone graft material to facilitate fusion between adjacent vertebrae. While retracted or shielded, the light detecting optode 1252 can detect back-scattering of NIR light emitted into the spinal cord S and output a detection signal to the computing device 1270 that represents such back-scattered NIR light for processing. For example, as discussed in more detail below with respect to FIG. 14, the computing device 1270 can be configured to monitor neural activity, status, health, etc. of the spinal cord S based on the detected back-scattering. The device 1200 can be used to retract and/or shield any of a variety of nerves, including exiting nerve roots, peripheral nerves, innervated tissue, and so forth.

Although FIGS. 12A and 12B illustrate a neural monitoring device 1200 including a hand-held retractor having a curved blade, it should be understood that embodiments of the neural monitoring device 1200 can also include other types of hand-held retractors, including but not limited to Hohmann retractors, Senn retractors, Army-Navy retractors, Ribbon retractors, Farabeuf retractors, Deaver retractors, and Richardson retractors.

For example, FIGS. 13A and 13B are schematic illustrations of a Hohmann retractor 1310 configured for neural monitoring. As shown, the Hohmann retractor 1310 can have a proximal handle portion 1310p and a distal retractor blade 1310d with at least one light emitting optode 1350 and at least one light detecting optode 1352 disposed on the retractor blade. As shown, the retractor blade 1310d can have a substantially planar proximal end portion 1312 and a tapered distal end portion 1314. The tapered distal end portion 1314 can be shaped in the form of a curved tip or hook configured for tissue or nerve retraction.

In some embodiments, the distal retractor blade 1310d, or a portion thereof, can be translucent to NIR light, such that the optodes 1350 and 1352 can be disposed on the blade in a manner that avoids direct contact with a nerve or innervated tissue during retraction. For example, as shown in FIGS. 13A and 13B, the light emitting optode 1350 and the light detecting optode 1352 can be disposed on a surface 1320 of the blade that opposes a contact surface 1322 of the blade. In some embodiments, the light emitting and light detecting optodes 1350, 1352 can be disposed or embedded between the first and second surfaces 1320, 1322, such that both optodes avoid direct contact with a retracted nerve or innervated tissue. As discussed above with respect to FIGS. 12A and 12B, the translucent retractor blade 1310d can allow NIR light to be emitted and reflected back through the blade between the optodes 1350, 1352 and the retracted nerve or tissue.

As shown in the illustrated embodiment FIG. 13C, the Hohmann retractor 1310 can be used for neural monitoring while retracting innervated tissue, such as psoas muscle 10, using a posterior approach. For example, the hand-held nerve retractor 1310 can be inserted through an incision made in the patient's side and distally advanced to the spine via a lateral approach. The retractor 1310 can be manipulated to retract the psoas muscle. While retracted, the light detecting optode 1352 can detect back-scattering of NIR light along an arcuate path P1 through the psoas muscle 10 and output a detection signal to the computing device 1270 that represents such back-scattered NIR light for processing. For example, as discussed in more detail below with respect to FIG. 14, the computing device 1270 can be configured to monitor neural activity, status, health, etc. of psoas nerves 30 within the retracted psoas muscle 10 based on the detected back-scattering.

FIG. 14 is a schematic illustration of an exemplary embodiment of a computing device 1400 that can be configured to perform neural monitoring using fNIR spectroscopy. The computing device 1400 can include various circuits and other electronic components used to power and control the operation of the computing device 1400. For example, as shown in the illustrated embodiment, the computing device 1400 can include a processor 1410, memory 1412, an optode I/O processor 1414, a display/audio output 1416, a network communications processor 1418, and a power supply 1420. In some embodiments, the processor 1410, the memory 1412, the optode I/O processor 1414, display/audio output 1416, the network communications processor 1418, and any other electronic components of the computing device 1400 may be powered by the power supply 1420. In some embodiments, the power supply 1420 may be a battery, a solar cell, or other type of energy harvesting power supply.

In some embodiments, the processor 1410 may be any programmable microprocessor, microcomputer, microcontroller, or multiple processor chip or chips that can be configured by software instructions (e.g., applications) to perform a variety of functions, including but not limited to controlling one or more light emitting optodes 1452 and light detecting optodes 1454 coupled to any of the retractors, docking ports, monitoring patches, or other surgical instruments shown and described in connection with FIGS. 2A-13B, determining neural activity, heath, status, etc. of a nerve or innervated tissue based on the output of a light detecting optode 1454, and issuing neural status reports or alerts based on the determined neural activity, status, health, etc. through a display or audio output 1416. The software instructions and/or software applications may be stored in the memory 1412 before they are accessed and loaded into the processor 1410. The processor 1410 may additionally or alternatively include internal memory sufficient to store such software instructions and/or applications.

The memory 1412 may be volatile memory (e.g., random access memory or RAM), non-volatile memory (e.g., flash memory), or a combination thereof. The memory 1412 may include internal memory included in the processor 1410, memory external to the processor 1410, or any combination thereof. The memory 1412 may store processor-executable instructions. In some embodiments, the memory 1412 can store output data generated by or associated with the light detecting optodes 1454 and/or light emitting optodes 1452 for use in analyzing the neural activity, status, health, etc. of a nerve or innervated tissue. In some embodiments, the memory 1412 can store data generated by the processor 1410 during analysis of the output data provided by the light detecting optodes 1454 and/or light emitting optodes 1452. In some embodiments, the memory 1412 can store neural status report or alerts generated by the processor 1410.

The optode I/O processor 1414 can be dedicated or programmable hardware specifically adapted to interface or communicate with the light emitting optodes 1452 and the light detecting optodes 1452. In some embodiments, the optode I/O processor 1414 can be configured to send commands or signals that can power on or otherwise activate one or more light emitting optodes 1452 and one or more light detecting optodes 1454 of the neural monitoring device 1450. In some embodiments, the optode I/O processor 1414 can be configured to send commands or signals to steer or adjust the directional orientation of a light emitting optode 1452 and/or a light detecting optode 1454 to target a specific nerve or region of innervated tissue. In some embodiments, the optode I/O processor 1414 can be configured to send commands or signals to cause a light emitting optode 1452 to emit NIR light at certain amplitudes, phases, and/or frequencies. In some embodiments, the optode I/O processor 1414 can be configured to receive an output signal that represents back-scattered NIR light detected by a light detecting optode 1454.

In some embodiments, transmission of one or more of the commands or signals sent to the optodes 1450, 1452 can be initiated in response to an internal command or signal received by the optode I/O processor 1414 from the processor 1410. In some embodiments, the functionality of the processor 1410 and the optode I/O processor 1414 can be incorporated into a single processor. In some embodiments, the optode I/O processor 1414 may be configured to interface or communicate directly with the light emitting optodes 1452 and the light detecting optodes 1454 of an embodiment neural monitoring device 1450 over a wired connection. In some embodiments, the optode I/O processor 1414 can be configured to interface or communicate with the optodes 1452, 1454 over a wireless link using a network communications processor 1418. In some embodiments, the network communications processor 1430 can be a two-way transceiver processor configured to transmit and receive signals over a wireless link using Bluetooth®, Wi-Fi® or other wireless communication protocol.

As previously discussed, the processor 1410 can be configured to determine or assess neural activity, status, health, etc. of a nerve or innervated tissue based on changes in the relative concentrations of oxygenated hemoglobin and deoxygenated hemoglobin detected in localized blood flow to the nerve or innervated tissue using fNIR spectroscopy. To determine the respective concentrations of oxygenated and deoxygenated hemoglobin of localized blood flow in a nerve or innervated tissue, fNIR spectroscopy can take advantage of the differences in the absorption of NIR light by oxygenated and deoxygenated hemoglobin above and below a wavelength of approximately 810 nm at which their respective absorption coefficients are the same (i.e., the isosbestic point). For example, oxygenated hemoglobin has larger absorption coefficients at wavelengths above the isosbestic point as compared to deoxygenated hemoglobin, and thus absorbs more light than deoxygenated hemoglobin at such wavelengths. Conversely, deoxygenated hemoglobin has larger absorption coefficients at wavelengths below the isosbestic point as compared to oxygenated hemoglobin, and thus absorbs more light than deoxygenated hemoglobin at such wavelengths.

Accordingly, in some embodiments, the processor 1410 can transmit one or more commands or signals, via the optode I/O processor 1430, that cause a light emitting optode 1452 to emit a first wavelength of NIR light above the isosbestic point of approximately 810 nm and a second wavelength of NIR light below the isosbestic point. In some embodiments, the light emitting optode 1450 can emit the respective wavelengths of NIR light simultaneously or sequentially. In some embodiments, the light emitting optode 1450 can include separate light emitting optodes, of which a first light emitting optode can be configured to emit NIR light above the isosbestic point and a second light emitting optode can be configured to emit NIR light below the isosbestic point.

As the emitted light is reflected or back-scattered along a path from the light emitting optode 1450 and detected by a light detecting optode, the intensity of the emitted light can become attenuated due, in part, to absorption by hemoglobin. For example, the intensity of the NIR light above the isosbestic point can become attenuated when absorbed by oxygenated hemoglobin along the path and the intensity of the NIR light below the isosbestic point can become attenuated when absorbed by deoxygenated hemoglobin along the path.

Accordingly, in some embodiments, the processor 1410 can receive a detection signal from the light detecting optode 1452 that represents the intensity of back-scattered NIR light at the first wavelength above the isosbestic point and the intensity of back-scattered NIR light at the second wavelength below the isosbestic point. In some embodiments, the light detecting optode 1452 can include separate light detecting optodes, of which a first light detecting optode outputs a first detection signals representing the detected intensity of back-scattered NIR light at the first wavelength and a second light detecting optode that outputs a second detection signal representing the detected intensity of back-scattered NIR light at the second wavelength.

In some embodiments, the processor 1410 can translate or convert detected changes in light intensity above and below the isosbestic point into respective changes in oxygenated and deoxygenated hemoglobin using the modified Beer-Lambert law or suitable conversion algorithm. For example, in some embodiments, the attenuation of light intensity after absorption and scattering by biological tissue can be expressed as:

I=GI₀e^{-(∝}^{_{HB}}^{C}^{_{HB}}^{+∝}^{_{HBO2}}^{C}^{_{HBO2}}^{)L}

where G is a factor that accounts for the measurement geometry and can be assumed constant when concentration changes. I₀ is input light intensity; ∝_{HB} and ∝_{HBO2} represent B and αHBO2 represent molar extension coefficients; C_{HB} and C_{HBO2} indicate concentrations of deoxygenated and oxygenated hemoglobin, respectively; and L represents the photon path, a function of absorption and scattering coefficients μₐ and μ_{b}. By measuring optical density (OD) changes at the two wavelengths, relative changes in oxygenated and deoxygenated hemoglobin concentration versus time can be obtained. If the intensity measurement at an initial time (baseline) is I_{b}, and at another time is I, the optical density change due to variation in deoxygenated hemoglobin concentration C_{HB} and oxygenated hemoglobin concentration C_{HBO2} during that period can be expressed as:

$$Δ OD = log ( \frac{{I}_{b}}{I} ) = {∝}_{HB} Δ {C}_{HB} + {∝}_{HBO 2} Δ {C}_{HBO 2} .$$

Measurements of ΔOD performed at the two different wavelengths allow the calculation of ΔC_{HB} and ΔC_{HBO2}. In some embodiments, oxygenation of the nerve or innervated tissue under analysis can be determined as the calculated difference between the variation in deoxygenated hemoglobin concentration ΔC_{HB} and the variation in oxygenated hemoglobin concentration ΔC_{HBO2} (ΔC_{HBO2}−ΔC_{HB}). Further details for converting detected changes in light intensity into respective changes in oxygenated and deoxygenated hemoglobin can be found in a publication entitled “Functional Near-Infrared Spectroscopy (fNIRS): Principles and Neuroscientific Applications” by José León-Carrión et al (2012), available from: http://www.intechopen.com/books/neuroimaging-methods/functional-near-infrared-spectroscopy-fnirs-brain-studies-and-others-clinical-uses, which is hereby incorporated herein by reference.

In some embodiments, the processor 1410 can transmit a command or signal to a display or audio output 1416 in response to the processor determining that a relative or absolute concentration of oxygenated hemoglobin in the localized blood flow to a targeted nerve or innervated tissue is below a threshold concentration or has been below the threshold concentration for a threshold period of time. In some embodiments, the threshold concentration and the threshold period of time can depend on the particular nerve or surgical procedure being performed. In some embodiments, the display/audio output 1416 can display or otherwise present information, such as a status report, that indicates an oxygenation state of a target nerve or innervated tissue and an amount of time during which the nerve or tissue has been in that state. In some embodiments, the display/audio output 1416 can display or output an alert notifying the surgeon to perform reperfusion (e.g., remove a tourniquet or retractor), and thereby increase the supply of oxygenated blood flow to the targeted nerve or innervated tissue, in response to the expiration of a timer that tracks how long a nerve or tissue has been in an ischemic state, e.g., as determined through an fNIR spectroscopic analysis.

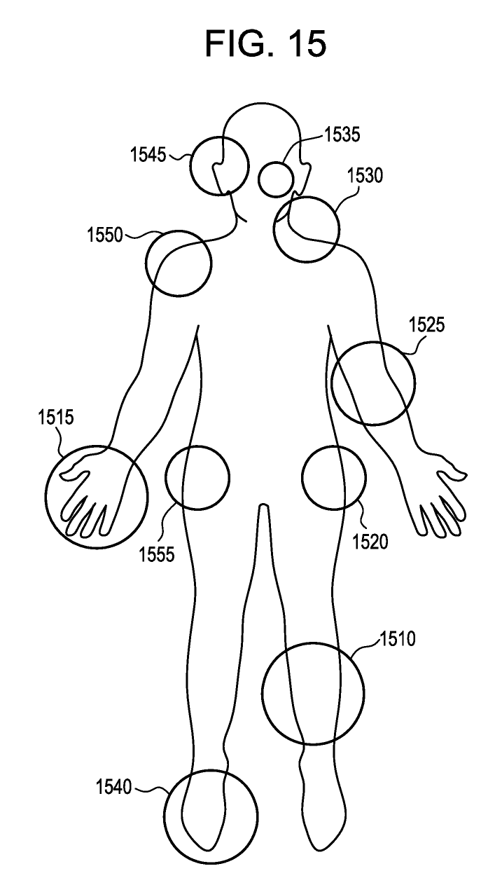

Although the FIGS. 2A-13C illustrate various embodiments of neural monitoring devices 200, 600, 700, 800, 900, and 1200 for use in spinal surgical procedures, it should be understood that any of the foregoing embodiment neural monitoring devices can be used in non-spinal surgical procedures. FIG. 15 is a schematic illustration identifying other exemplary parts of the body 1500 in which the various embodiments of the neural monitoring devices can be used.

In some embodiments, one or more of the embodiment neural monitoring devices 200, 600, 700, 800, 900, and 1200 can be used monitor neural activity, status, health, etc. of nerves and innervated tissue in the leg 1510. For example, during free fibular harvesting or flap harvesting, there can be a risk of neural damage to the peroneal nerve in the lower leg due to the tourniquet-induced ischemia and/or retraction of innervated tissue, such as foot drop. In some embodiments, one or more of the embodiment neural monitoring devices 200, 600, 700, 800, 900, and 1200 can be used to track the length of time that such nerves are in an ischemic state, e.g., as determined through an fNIR spectroscopic analysis, and alert the surgeon through an display or audible output device after a timer expires indicating that the tourniquet or retraction should be removed to facilitate reperfusion of the nerve.

In some embodiments, one or more of the embodiment neural monitoring devices 200, 600, 700, 800, 900, and 1200 can be used monitor neural activity, status, health, etc. of nerves and innervated tissue in the hand 1515. For example, during endoscopic carpal tunnel release procedures, there can be a risk of neural damage to the recurrent motor branch and/or the medial nerve due to excessive retraction of those nerves. In other hand surgeries, the surgeon may exsanguinate an elevated hand and then apply a tourniquet proximate to the hand to prevent blood flow to the hand. Although the tourniquet can reduce bleeding and facilitate faster surgery times, the nerves generally in the hand are ischemic due to the lack of oxygenated blood flow. In some embodiments, one or more of the embodiment neural monitoring devices 200, 600, 700, 800, 900, and 1200 can be used track the length of time that such nerves are in an ischemic state, e.g., as determined through an fNIR spectroscopic analysis, and alert the surgeon through an display or audible output device after a timer expires indicating that the tourniquet or retraction should be removed to facilitate reperfusion of the nerve.

In some embodiments, one or more of the embodiment neural monitoring devices 200, 600, 700, 800, 900, and 1200 can be used monitor neural activity, status, health, etc. of nerves and innervated tissue in the hip 1520. For example, during revision hip surgery, there can be a risk of neural damage to the sciatic nerve due to dissection and excessive retraction of the tissue containing that nerve. In particular, a surgeon may dissect the sciatic nerve away from the hip and retract it to position that does not interfere with the surgeon's access to the surgical site. In some embodiments, one or more of the embodiment neural monitoring devices 200, 600, 700, 800, 900, and 1200 can be used to track the length of time that the sciatic nerve is in an ischemic state, e.g., as determined through an fNIR spectroscopic analysis, and alert the surgeon through an display or audible output device after a timer expires indicating that the retraction should be removed to facilitate reperfusion of the nerve.

In some embodiments, one or more of the embodiment neural monitoring devices 200, 600, 700, 800, 900, and 1200 can be used monitor neural activity, status, health, etc. of nerves and innervated tissue in the elbow 1525. For example, during total elbow replacement or complex elbow fractures, there can be a risk of neural damage to the ulnar nerve due to dissection and excessive retraction of the nerve. In particular, a surgeon may dissect the ulnar nerve out of the elbow and retract it to position that does not interfere with the surgeon's access to the surgical site. In some embodiments, one or more of the embodiment neural monitoring devices 200, 600, 700, 800, 900, and 1200 can be used to track the length of time that the ulnar nerve is in an ischemic state, e.g., as determined through an fNIR spectroscopic analysis, and alert the surgeon through an display or audible output device after a timer expires indicating that the retraction should be removed to facilitate reperfusion of the nerve.

In some embodiments, one or more of the embodiment neural monitoring devices 200, 600, 700, 800, 900, and 1200 can be used monitor neural activity, status, health, etc. of nerves and innervated tissue at or near the neck 1530. For example, during a thyroidectomy, a surgeon may create a surgical path to the thyroid starting from at an incision made below the thyroid (e.g. at or about the clavicle). During such procedures, there can be a risk of neural damage to the recurrent laryngeal nerve that is in the surgical field for thyroidectomy. To avoid such damage, in some embodiments, one or more optodes 1250 and counterpart optodes 1252 disposed at the distal end of a guide wire, catheter, stylet or other elongated instrument (e.g., 1210) of the embodiment neural monitor device 1200 can be used to monitor the oxygenation levels of the recurrent laryngeal nerve or other peripheral nerve encountered along the surgical path as the instrument is inserted from the incision to the thyroid.

In some embodiments, one or more the embodiment neural monitoring devices 200, 600, 700, 800, 900, and 1200 can be used monitor neural activity, status, health, etc. of nerves and innervated tissue during trans-orifice surgery, abdominal surgery, or other surgical approaches in which the optode and counterpart optodes can be deployed within the patient through a natural orifice (e.g., mouth 1535, etc.) without making an incision through the skin.

In some embodiments, one or more the embodiment neural monitoring devices 200, 600, 700, 800, 900, and 1200 can be used monitor neural activity, status, health, etc. of nerves and innervated tissue in the foot 1540 or other lower extremity. For example, diabetic patients can experience foot problems due to loss of sensation from diabetic neuropathy, ulcers, and the like. Neuropathy occurs slowly over long period of time. Accordingly, in some embodiments, the neural monitoring patch 810 of the neural monitoring device 800 can be applied to the outer skin surface of a patient's foot or other lower extremity to monitor oxygenation levels to prevent potential neural damage or impairment.

In some embodiments, one or more of the embodiment neural monitoring devices 200, 600, 700, 800, 900, and 1200 can be used monitor neural activity, status, health, etc. of nerves and innervated tissue in the face 1545, such as the facial nerve and parotid gland. For example, the facial nerve supplies motor signals to every part of the face 1520 except for a few chewing muscles. It can be challenging to dissect the parotid gland because about ninety percent of the muscles in and around the face 1520 are innervated by the facial nerve (i.e., seventh cranial nerve). Thus, in some embodiments, one or more of the embodiment neural monitoring devices 200, 600, 700, 800, 900, and 1200 can be used to continuously monitor neural activity, status, health, etc. of the facial nerve based on relative concentrations of oxygenated and deoxygenated hemoglobin in blood flow to the facial nerve in the face 1520.

In some embodiments, one or more of the embodiment neural monitoring devices 200, 600, 700, 800, 900, and 1200 can be used monitor neural activity, status, health, etc. of nerves located in any innervated tissue in which dissection or resection can be challenged by nerves extending there through. For example, in some embodiments, one or more of the embodiment neural monitoring devices 200, 600, 700, 800, 900, and 1200 can be used to track the length of time that the brachial plexus in the shoulder 1550 or lumbosacral plexus in the hip 1555 is in an ischemic state, e.g., as determined through an fNIR spectroscopic analysis, and alert the surgeon through an display or audible output device after a timer expires indicating that reperfusion of the nerves should be initiated.

Additional details regarding fNIR spectroscopy can be found in the following references:
Brink, Frontiers in Psychology, April 2001, 2, 80, 1-16, the entire contents of which are incorporated herein by reference
José León-Carrión, et al. (2012). Functional Near-Infrared Spectroscopy (fNIRS): Principles and Neuroscientific Applications. Neuroimaging—Methods. Prof. Peter Bright (Ed.), ISBN: 978-953-51-0097-3, the entire contents of which are incorporated herein by reference.
Murkin, J. M., et al. (2009). Near-infrared spectroscopy as an index of brain and tissue oxygenation. British Journal of Anaesthesia 103(BJA/PGA Supplement), pp. i3-i13, the entire contents of which are incorporated herein by reference.
Kroczek, Agnes, et al. (2015). Prefrontal functional connectivity measured with near-infrared spectroscopy during smoking cue exposure. Addiction Biology, 22(2), the entire contents of which are incorporated herein by reference.
Glassman Lisa H., et al. (2016). The relationship between dorsolateral prefrontal activation and speech performance-based social anxiety using functional near infrared spectroscopy. Brain Imaging and Behavior. 11(3), pp. 797-807, the entire contents of which are incorporated herein by reference.
Khan, Bilal, et al. (2011). Spatiotemporal relations of primary sensorimotor and secondary motor activation patterns mapped by NIR imaging. Biomedial Optics Express. 2(12), pp. 3367-3386, the entire contents of which are incorporated herein by reference.
Kokan, Norio, et al. (2011). Near-infrared spectroscopy of orbitofrontal cortex during odorant stimulation. American Journal of Rhinology & Allergy, 25(3), pp. 163-165, the entire contents of which are incorporated herein by reference.
McKendrick, Ryan, et al. (2015). Wearable functional near infrared spectroscopy (fNIRS) and transcranial direct current stimulation (tDCS): expanding vistas for neurocognitive augmentation. Frontiers in Systems Neuroscience, 9(27), pp. 1-14, the entire contents of which are incorporated herein by reference.
Germon, T. J., et al. (1999) Cerebral near infrared spectroscopy: emitter-detector separation must be increased. British Journal of Anaesthesia, 82(6), pp. 831-837, the entire contents of which are incorporated herein by reference.
U.S. patent application Ser. No. 13/687,700, filed on Nov. 28, 2012, entitled “Functional Near Infrared Spectroscopy Imaging System and Method” and published as U.S. Publication No. 2013/0090541, the entire contents of which are incorporated herein by reference.
U.S. patent application Ser. No. 12/571,145, filed on Sep. 30, 2009, entitled “Functional near-infrared spectroscopy as a monitor for depth of anesthesia” and issued as U.S. Pat. No. 8,798,701, the entire contents of which are incorporated herein by reference.
Ferrari, Marco, et al. (2011). The use of near-infrared spectroscopy in understanding skeletal muscle physiology: recent developments. Philosophical Transactions of The Royal Society, 369, pp. 4577-4590, the entire contents of which are incorporated herein by reference.
Davis, Michelle L., et al. (2013). Estimated Contribution of Hemoglobin and Myoglobin to Near Infrared Spectroscopy. Respiratory Physiology & Neurobiology, 182(2), pp. 180-187, the entire contents of which are incorporated herein by reference.

It should be noted that any ordering of method steps expressed or implied in the description above or in the accompanying drawings is not to be construed as limiting the disclosed methods to performing the steps in that order. Rather, the various steps of each of the methods disclosed herein can be performed in any of a variety of sequences. In addition, as the described methods are merely exemplary embodiments, various other methods that include additional steps or include fewer steps are also within the scope of the present invention.

The devices disclosed herein and the various component parts thereof can be constructed from any of a variety of known materials. Exemplary materials include those which are suitable for use in surgical applications, including metals such as stainless steel, titanium, or alloys thereof, polymers such as PEEK, ceramics, carbon fiber, and so forth. The various components of the devices disclosed herein can be rigid or flexible. One or more components or portions of the device can be formed from a radiopaque material to facilitate visualization under fluoroscopy and other imaging techniques, or from a radiolucent material so as not to interfere with visualization of other structures. Exemplary radiolucent materials include carbon fiber and high-strength polymers.

In any of the foregoing embodiments, the light emitting optodes (or light emitters) can be lasers, light emitting diodes, or other device suitable for emitting light at a desired wavelength or within a desired range of wavelengths. The light detecting optodes (or light detectors) can be photodetectors or other suitable device for detecting an intensity, phase, or frequency of light at a desired wavelength or within desired range of wavelengths.

The systems and methods disclosed herein can be used in minimally-invasive surgery and/or open surgery. While the systems and methods disclosed herein are generally described in the context spinal surgery, it will be appreciated that the systems and methods disclosed herein can be used with any human or animal in any of a variety of surgeries performed on humans or animals. While devices and methods that employ NIR light are generally described herein, it will be appreciated that the emitters and detectors can operate with different kinds of light, radiation, sound, etc.

Although specific embodiments are described above, it should be understood that numerous changes may be made within the spirit and scope of the concepts described. Accordingly, it is intended that this disclosure not be limited to the described embodiments, but that it have the full scope defined by the language of the following claims.

1-9. (canceled)

10. A device for neural monitoring, comprising:
a processor;
an access device having a proximal end and a distal end and defining a working channel into a body of a patient; and
one or more near infrared (NIR) light emitters and one or more NIR light detectors disposed on the distal end of the access device,
wherein the one or more NIR light emitters are configured to emit NIR light and the one or more NIR light detectors are configured to detect back-scattered NIR light, and
wherein the processor is configured to perform neural monitoring based on the detected back-scattered NIR light reflected from the nerve.

11. The device of claim 10, wherein the processor is configured to determine relative concentrations of oxygenated hemoglobin and deoxygenated hemoglobin based on the detected back-scattered NIR light and monitor neural status of the nerve based on the determined concentrations.

12. The device of claim 10, wherein at least one of the one or more NIR light emitters is configured to emit collimated NIR light into the nerve.

13. The device of claim 12, wherein at least one of the one or more NIR light detectors is housed within an open-ended tube through which to detect the collimated NIR light reflected from the nerve.

14. The device of claim 10, wherein the one or more NIR light detectors comprise a plurality of NIR light detectors oriented to detect the back-scattered NIR light reflected from the nerve in different directions.

15. The device of claim 14, wherein the processor is configured to selectively activate one or more of the plurality of NIR light detectors to detect back-scattered NIR light reflected from the nerve in one or more of the different directions.

16. The device of claim 10, wherein the one or more NIR light emitters comprises a plurality of light emitters oriented to emit NIR light towards the nerve in different directions.

17. The device of claim 16, wherein the processor is configured to selectively activate one or more of the plurality of NIR light emitters to emit the NIR light towards the nerve in one or more of the different directions.

18. The device of claim 10, wherein the access device is a substantially tubular docking port.

19. The device of claim 10 wherein at least one of the NIR light emitters is steerable to emit the NIR light towards a nerve and at least one of the NIR light detectors is steerable to detect the back-scattered NIR light reflected from the nerve.

20-24. (canceled)

25. A device for neural monitoring, comprising:
a processor;
an access device having a proximal end and a distal end and defining a working channel into a body of a patient;
an elongated instrument having a proximal end and a distal end, wherein the elongated instrument is configured to be inserted through the working channel of the access device into the body of the patient;
one or more optodes disposed on the distal end of the access device; and
one or more counterpart optodes disposed on the distal end of the elongated instrument;
wherein the one or more optodes or the one or more counterpart optodes are configured to emit near infrared (NIR) light and wherein the one or more counterpart optodes or the one or more optodes are configured to detect back-scattered NIR light,
wherein the elongated instrument is distally advanced or withdrawn with respect to the distal end of the access device to vary a distance between the one or more optodes and the one or more counterpart optodes and thereby change the maximum depth from which to detect the back-scattered NIR light, and
wherein the processor is coupled to the one or more optodes and the one or more counterpart optodes and configured to perform neural monitoring based on the detected back-scattered NIR light.

26. The device of claim 25, wherein the processor is configured to determine relative concentrations of oxyhemoglobin hemoglobin and deoxyhemoglobin hemoglobin based on the detected back-scattered NIR light and monitor neural status based on the determined concentrations.

27. The device of claim 25, wherein the one or more optodes comprises one or more NIR light emitters and the one or more counterpart optodes comprises one or more NIR light detectors.

28. The device of claim 25, wherein the one or more counterpart optodes comprises one or more NIR light emitters and the one or more optodes comprises one or more NIR light detectors.

29. The device of claim 25, wherein the access device is a substantially tubular docking port.

30. The device of claim 25, wherein the elongated instrument is a nerve retractor having a retractor blade at the distal end.

31. The device of claim 30, wherein the retractor blade has a contact surface and a non-contact surface that opposes the contact surface of the retractor blade, the contact surface of the retractor blade being configured to contact a nerve or innervated tissue, and
wherein the retractor blade comprises a translucent portion between the contact surface and the non-contact surface of the blade that is translucent to NIR light.

32. The device of claim 31, wherein one of the contact surface and the non-contact surface of the retractor blade is convex and one of the contact surface and the non-contact surface of the retractor blade is concave.

33. The device of claim 31 wherein the one or more counterpart optodes is one or more NIR light emitters disposed on the non-contact surface of the retractor blade such that the NIR light is emitted from the one or more NIR light emitters through the translucent portion and the contact surface of the retractor blade.

34. The device of claim 31 wherein the one or more counterpart optodes is one or more NIR light detectors disposed on the non-contact surface of the retractor blade such that the back-scattered NIR light entering through the contact surface and the translucent portion of the retractor blade is detected by the one or more NIR light detectors.

35. The device of claim 31, wherein the one or more counterpart optodes are disposed in the translucent portion of the retractor blade between the contact surface and the non-contact surface of the blade.

36. The device of claim 25, wherein the elongated instrument is a guide wire, stylet, cannula, catheter, probe, or nerve shield.

37-43. (canceled)

44. A method for neural monitoring, comprising:
inserting an access device at least partially in an incision or a natural orifice of a patient, wherein the access device defines a working channel having a proximal end and a distal end, wherein one or more near infrared (NIR) light emitters and one or more NIR light detectors disposed on the distal end of the access device;
emitting near infrared (NIR) light by at least one of the NIR light emitters;
outputting a signal by at least one of the NIR light detectors that is indicative of the NIR light reflected along a path thereto; and
monitoring a neural status of a targeted nerve or innervated tissue based on the signal indicative of the NIR light reflected along the path passing through the target nerve or innervated tissue.

45. The method of claim 44, wherein monitoring the neural status of the targeted nerve or innervated tissue based on the signal comprises determining based on the signal relative concentrations of oxygenated hemoglobin and deoxygenated hemoglobin associated with the targeted nerve or innervated tissue and to determine the neural status of the targeted nerve or innervated tissue based on the determined concentrations.

46. The method of claim 44, wherein the one or more NIR light detectors comprises a plurality of NIR light detectors oriented to receive NIR light reflected along different paths and the method further comprises selectively activating one or more of the plurality of NIR light detectors to receive NIR light reflected along one or more of the different paths.

47. The method of claim 44, wherein the one or more NIR light emitters comprises a plurality of light emitters oriented to emit the NIR light along different paths and the method further comprises selectively activating one or more of the plurality of NIR light emitters to emit NIR light along one or more of the different paths.

48. The method of claim 44, wherein the access device is a substantially tubular docking port.

49. The method of claim 44, further comprising controlling a direction of at least one of the one or more NIR light emitters and at least one of the one or more NIR light detectors to adjust the path of the NIR light reflected therebetween to pass through a targeted nerve or innervated tissue.

50-54. (canceled)

55. A method for neural monitoring, comprising:
inserting an access device at least partially in an incision or a natural orifice of a patient, wherein the access device defines a working channel having a proximal end and a distal end, wherein one or more optodes are disposed on the distal end of the access device;
inserting a distal end of an elongated instrument through working channel of the access device and below the distal end of the access device, wherein one or more counterpart optodes are disposed on the distal end of the elongated instrument;
emitting near infrared (NIR) light by at least one of the optodes or counterpart optodes;
outputting a signal by at least one of the counterpart optodes or optodes that is indicative of the NIR light reflected along a path thereto;
advancing or withdrawing the elongated instrument below the distal end of the access device, such that the path of the NIR light between the at least one optode disposed on the access device and the at least one counterpart optode disposed on the elongated instrument is adjusted to pass through a targeted nerve or innervated tissue; and
monitoring neural status of the targeted nerve or innervated tissue based on the signal indicative of the NIR light reflected along the path passing through the targeted nerve or innervated tissue.

56. The method of claim 55, wherein the one or more optodes comprises one or more NIR light emitters and the one or more counterpart optodes comprises one or more NIR light detectors.

57. The method of claim 55, wherein the one or more counterpart optodes comprises one or more NIR light detectors and the one or more counterpart optodes comprises one or more NIR light emitters.

58. The method of claim 55, wherein monitoring neural status of the targeted nerve or innervated tissue based on the signal comprises:
determining based on the signal relative concentrations of oxygenated hemoglobin or deoxygenated hemoglobin associated with the targeted nerve or innervated tissue; and
determining the neural status associated with the targeted nerve or innervated tissue based on the determined concentrations.

59. The method of claim 55, wherein the elongated instrument includes a guide wire, stylet, cannula, catheter, probe, nerve retractor, or nerve shield.

60. The method of claim 55, wherein the access device is a substantially tubular docking port.

61. (canceled)

62. The method of claim 44, wherein the targeted nerve or innervated tissue is located at or adjacent to one or more of the spine, leg, hip, hand, shoulder, face, neck, elbow, and foot.

63. (canceled)

64. The method of claim 54, wherein the targeted nerve or innervated tissue is located at or adjacent to one or more of the spine, leg, hip, hand, shoulder, face, neck, elbow, and foot.
